SEQUENCE LISTING (44 sequences)

SEQ ID NO: 1 - protein, 20 aa, synthetic construct
  REGION 1..20: note = Membrane trafficking signal
KSRITSEGEYIPLDQIDINV

SEQ ID NO: 2 - protein, 7 aa, synthetic construct
  REGION 1..7: note = ER EXPORT SIGNAL
FCYENEV

SEQ ID NO: 3 - protein, 9 aa, synthetic construct
  REGION 1..9: note = Rhodopsin epitope tag
TETSQVAPA

SEQ ID NO: 4 - protein, 6 aa, synthetic construct
  REGION 1..6: note = Linker sequence
PRARDP

SEQ ID NO: 5 - protein, 607 aa, synthetic construct
  REGION 1..607: note = eOPN3-mScarlet open reading frame AA sequence
MYDAPNDVASSVADYEDLMAPWAYNAAAITLFFIGFFGFFLNLFVIALMSKDMQLWTPMNIILFNLVCSDFSVSIIGNPLTLTSAISHRWIFGRTLCVAYGFFMSLLGITSITTLTVLSYERYCLISRPFSSRNLSRKGAFLAIFFIWGYSFALTSPPLFGWGAYVQEAANISCSVNWESQTKNATTYIIFLFVFGLVVPLIVIVYSYTNIIVYMRRNSARVGRINRAEQRVTSMVAVMIVAFMVAWTPYAIFALIEQFGPPELIGPGLAVLPALIAKSSICYNPIIYVGMNTQFRAAFTRVRNKGGVPTADQNTTTMQRELTKSSRDMVTETSQVAPAPRARDPTGKSRITSEGEYIPLDQIDINVVMVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYKFCYENEV

SEQ ID NO: 6 - DNA, 1824 bp, synthetic construct
  misc_feature 1..1824: note = eOPN3 nucleic acid sequence
  source 1..1824: mol_type = other DNA
atgtacgacgcccccaacgacgtggccagcagcgtggccgactacgaggacctgatggccccctgggcctacaacgccgccgccatcaccctgttcttcatcggcttcttcggcttcttcctgaacctgttcgtgatcgccctgatgagcaaggacatgcagctgtggacccccatgaacatcatcctgttcaacctggtgtgcagcgacttcagcgtgagcatcatcggcaaccccctgaccctgaccagcgccatcagccacaggtggatcttcggcaggaccctgtgcgtggcctacggcttcttcatgagcctgctgggcatcaccagcatcaccaccctgaccgtgctgagctacgagaggtactgcctgatcagcaggcccttcagcagcaggaacctgagcaggaagggcgccttcctggccatcttcttcatctggggctacagcttcgccctgaccagcccccccctgttcggctggggcgcctacgtgcaggaggccgccaacatcagctgcagcgtgaactgggagagccagaccaagaacgccaccacctacatcatcttcctgttcgtgttcggcctggtggtgcccctgatcgtgatcgtgtacagctacaccaacatcatcgtgtacatgaggaggaacagcgccagggtgggcaggatcaacagggccgagcagagggtgaccagcatggtggccgtgatgatcgtggccttcatggtggcctggaccccctacgccatcttcgccctgatcgagcagttcggcccccccgagctgatcggccctggactggctgtgctgcctgctctgatcgccaagagcagcatctgctacaaccccatcatctacgtgggcatgaacacccagttcagggccgccttcaccagggtgaggaacaagggcggcgtgcccaccgccgaccagaacaccaccaccatgcagagggagctgaccaagagcagcagggacatggtgaccgagaccagccaggtggctcctgctccgcgagcccgagatccaaccggtaagagcaggatcaccagcgagggcgagtacatccccctggaccagatcgacatcaacgtggtgatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagttctgctacgagaacgaggtgtaa

SEQ ID NO: 7 - DNA, 696 bp, synthetic construct
  misc_feature 1..696: note = mScarlet NA sequence
  source 1..696: mol_type = other DNA
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaag

SEQ ID NO: 8 - protein, 429 aa, synthetic construct
  REGION 1..429: note = mosopn3 aa sequence
MYDAPNDVASSVADYEDLMAPWAYNAAAITLFFIGFFGFFLNLFVIALMSKDMQLWTPMNIILFNLVCSDFSVSIIGNPLTLTSAISHRWIFGRTLCVAYGFFMSLLGITSITTLTVLSYERYCLISRPFSSRNLSRKGAFLAIFFIWGYSFALTSPPLFGWGAYVQEAANISCSVNWESQTKNATTYIIFLFVFGLVVPLIVIVYSYTNIIVYMRRNSARVGRINRAEQRVTSMVAVMIVAFMVAWTPYAIFALIEQFGPPELIGPGLAVLPALIAKSSICYNPIIYVGMNTQFRAAFTRVRNKGGVPTADQNTTTMQRELTKSSRDMVECSFDFCRKKNRFKISLVKPTAPLAVVDVSSSSHPGKVTSRSPLDQTVLNEMNDEERGRERSGAGYAGSRFVRPDFELSVINSGKSILIKSKNFRSNLL

SEQ ID NO: 9 - protein, 330 aa, synthetic construct
  REGION 1..330: note = mosopn3 mutant without the last 99 aa
MYDAPNDVASSVADYEDLMAPWAYNAAAITLFFIGFFGFFLNLFVIALMSKDMQLWTPMNIILFNLVCSDFSVSIIGNPLTLTSAISHRWIFGRTLCVAYGFFMSLLGITSITTLTVLSYERYCLISRPFSSRNLSRKGAFLAIFFIWGYSFALTSPPLFGWGAYVQEAANISCSVNWESQTKNATTYIIFLFVFGLVVPLIVIVYSYTNIIVYMRRNSARVGRINRAEQRVTSMVAVMIVAFMVAWTPYAIFALIEQFGPPELIGPGLAVLPALIAKSSICYNPIIYVGMNTQFRAAFTRVRNKGGVPTADQNTTTMQRELTKSSRDMV

SEQ ID NO: 10 - protein, 288 aa, synthetic construct
  REGION 1..288: note = PufTMT amino acid sequence
MSRTGHTVVAVMLGTILLAGVFGNSVVFLVFVKYRSLRTPINLILLNISLSDILVCVFGTPLSFAASLKGRWLLGERGCEWYGFANSLFGIVSLVSLSVLSYERYTVVLQPTQVDVSYFRKAWFCVGGSWLYALFWTLPPLLGWSRYGPEGPGTMCSVQWHLRSPANISYVLCLFIFCLLLPLVVMVYSYGRIWVAVRRQHCAQSHLEAGRINLLTAQRREQHILWMVLSMVSCYMLCWMPYGIIALVATLGRLGPISPAVSVVPSILAKFSTVVNPVIYMFFNNQVR

SEQ ID NO: 11 - protein, 6 aa, synthetic construct
  REGION 1..6: note = ER EXPORT SIGNAL
  VARIANT 2: note = Xaa- can be any naturally occurring amino acid
FXYENE

SEQ ID NO: 12 - skipped

SEQ ID NO: 13 - protein, 5 aa, synthetic construct
  REGION 1..5: note = ER EXPORT SIGNAL
VKESL

SEQ ID NO: 14 - protein, 5 aa, synthetic construct
  REGION 1..5: note = ER EXPORT SIGNAL
VLGSL

SEQ ID NO: 15 - protein, 16 aa, synthetic construct
  REGION 1..16: note = ER EXPORT SIGNAL
NANSFCYENEVALTSK

SEQ ID NO: 16 - protein, 26 aa, synthetic construct
  REGION 1..26: note = MEMBRANE TRAFFICKING SIGNAL
MDYGGALSAVGRELLFVTNPVVVNGS

SEQ ID NO: 17 - protein, 26 aa, synthetic construct
  REGION 1..26: note = MEMBRANE TRAFFICKING SIGNAL
AGHSNSMALFSFSLLWLCSGVLGTEF

SEQ ID NO: 18 - protein, 23 aa, synthetic construct
  REGION 1..23: note = MEMBRANE TRAFFICKING SIGNAL
MGLRALMLWLLAAAGLVRESLQG

SEQ ID NO: 19 - protein, 18 aa, synthetic construct
  REGION 1..18: note = MEMBRANE TRAFFICKING SIGNAL
MRGTPLLLVVSLFSLLQD

SEQ ID NO: 20 - DNA, 864 bp, synthetic construct
  misc_feature 1..864: note = Codon optimized nucleic acid sequence of the MosOpn3
  source 1..864: mol_type = other DNA
atgagcaggaccggccacaccgtggtggccgtgatgctgggcaccatcctgctggccggcgtgttcggcaacagcgtggtgttcctggtgttcgtgaagtacaggagcctgaggacccccatcaacctgatcctgctgaacatcagcctgagcgacatcctggtgtgcgtgttcggcacccccctgagcttcgccgccagcctgaagggcaggtggctgctgggcgagaggggctgcgagtggtacggcttcgccaacagcctgttcggcatcgtgagcctggtgagcctgagcgtgctgagctacgagaggtacaccgtggtgctgcagcccacccaggtggacgtgagctacttcaggaaggcctggttctgcgtgggcggcagctggctgtacgccctgttctggaccctgcctcctctgctgggctggagcaggtacggccccgagggccctggcaccatgtgcagcgtgcagtggcacctgaggagccccgccaacatcagctacgtgctgtgcctgttcatcttctgcctgctgctgcccctggtggtgatggtgtacagctacggcaggatctgggtggccgtgaggaggcagcactgcgcccagagccacctggaggccggcaggatcaacctgctgaccgcccagaggagggagcagcacatcctgtggatggtgctgagcatggtgagctgctacatgctgtgctggatgccctacggcatcatcgccctggtggccaccctgggcaggctgggccccatcagccccgccgtgagcgtggtgcccagcatcctggccaagttcagcaccgtggtgaaccccgtgatctacatgttcttcaacaaccaggtgagg

SEQ ID NO: 21 - DNA, 990 bp, synthetic construct
  misc_feature 1..990: note = Alternative Codon optimized nucleic acid sequence of the MosOpn3
  source 1..990: mol_type = other DNA
atgtacgacgcccccaacgacgtggccagcagcgtggccgactacgaggacctgatggccccctgggcctacaacgccgccgccatcaccctgttcttcatcggcttcttcggcttcttcctgaacctgttcgtgatcgccctgatgagcaaggacatgcagctgtggacccccatgaacatcatcctgttcaacctggtgtgcagcgacttcagcgtgagcatcatcggcaaccccctgaccctgaccagcgccatcagccaccgctggatcttcggccgcaccctgtgcgtggcctacggcttcttcatgagcctgctgggcatcaccagcatcaccaccctgaccgtgctgagctacgagcgctactgcctgatcagccgccccttcagcagccgcaacctgagccgcaagggcgccttcctggccatcttcttcatctggggctacagcttcgccctgaccagcccccccctgttcggctggggcgcctacgtgcaggaggccgccaacatcagctgcagcgtgaactgggagagccagaccaagaacgccaccacctacatcatcttcctgttcgtgttcggcctggtggtgcccctgatcgtgatcgtgtacagctacaccaacatcatcgtgtacatgcgccgcaacagcgcccgcgtgggccgcatcaaccgcgccgagcagcgcgtgaccagcatggtggccgtgatgatcgtggccttcatggtggcctggaccccctacgccatcttcgccctgatcgagcagttcggcccccccgagctgatcggccccggcctggccgtgctgcccgccctgatcgccaagagcagcatctgctacaaccccatcatctacgtgggcatgaacacccagttccgcgccgccttcacccgcgtgcgcaacaagggcggcgtgcccaccgccgaccagaacaccaccaccatgcagcgcgagctgaccaagagcagccgcgacatggtg

SEQ ID NO: 22 - DNA, 864 bp, synthetic construct; identical to SEQ ID NO: 20
  misc_feature 1..864: note = Codon optimized nucleic acid sequence of the PufTMT
  source 1..864: mol_type = other DNA

SEQ ID NO: 23 - DNA, 864 bp, synthetic construct
  misc_feature 1..864: note = Alternative Codon optimized nucleic acid sequence of the PufTMT
  source 1..864: mol_type = other DNA
atgagccgcaccggccacaccgtggtggccgtgatgctgggcaccatcctgctggccggcgtgttcggcaacagcgtggtgttcctggtgttcgtgaagtaccgcagcctgcgcacccccatcaacctgatcctgctgaacatcagcctgagcgacatcctggtgtgcgtgttcggcacccccctgagcttcgccgccagcctgaagggccgctggctgctgggcgagcgcggctgcgagtggtacggcttcgccaacagcctgttcggcatcgtgagcctggtgagcctgagcgtgctgagctacgagcgctacaccgtggtgctgcagcccacccaggtggacgtgagctacttccgcaaggcctggttctgcgtgggcggcagctggctgtacgccctgttctggaccctgccccccctgctgggctggagccgctacggccccgagggccccggcaccatgtgcagcgtgcagtggcacctgcgcagccccgccaacatcagctacgtgctgtgcctgttcatcttctgcctgctgctgcccctggtggtgatggtgtacagctacggccgcatctgggtggccgtgcgccgccagcactgcgcccagagccacctggaggccggccgcatcaacctgctgaccgcccagcgccgcgagcagcacatcctgtggatggtgctgagcatggtgagctgctacatgctgtgctggatgccctacggcatcatcgccctggtggccaccctgggccgcctgggccccatcagccccgccgtgagcgtggtgcccagcatcctggccaagttcagcaccgtggtgaaccccgtgatctacatgttcttcaacaaccaggtgcgc

SEQ ID NO: 24 - protein, 5 aa, synthetic construct
  REGION 1..5: note = repetetive amino acid sequence motif
EAAAK

SEQ ID NO: 25 - protein, 354 aa, synthetic construct
  REGION 1..354: note = PdCO2 aa sequence
MDGENLTIPNPVTELMDTPINSTYFQNLNAETDGGNHYIYNAFTATDYNICAAYLFFIACLGVSLNVLVLVLFIKDRKLRSPNNFLYVSLALGDLLVAVFGTAFKFIITARKTLLREEDGFCKWYGFITYLGGLAALMTLSVIAFVRCLAVLRLGSFTGLTTRMGVAAMAFIWIYSLAFTLAPLLGWNHYIPEGLATWCSIDWLSDETSDKSYVFAIFIFCFLVPVLIIVVSYGLIYDKVRKVAKTGGSVAKAEREVLRMTLLMVSLFMLAWSPYAVICMLASFGPKDLLHPVATVIPAMFAKSSTMYNPLIYVFMNKQFRRSLKVLLGMGVEDLNSESERATGGTATNQVAAT

SEQ ID NO: 26 - DNA, 1062 bp, synthetic construct
  misc_feature 1..1062: note = PdCO2 na sequence
  source 1..1062: mol_type = other DNA
atggacggggagaacttgacgattccaaatcctgtcaccgagttgatggataccccaatcaacagcacgtatttccaaaaccttaatgcggaaacggacggcggaaatcattacatctacaatgccttcacagccaccgattacaacatttgtgcagcttaccttttcttcattgcttgtctgggagtcagtctcaacgttttggtgttggtcctctttatcaaagacagaaagttgaggagccccaataacttcctgtatgtaagtctcgctctcggggatctgttggttgcagttttcggcaccgcatttaagttcattattacagctcgcaaaaccctgttgcgagaagaagacggtttctgcaaatggtacggcttcataacatacctcggcgggctcgcggcactgatgaccctgagcgttattgcttttgttcgttgtctggccgtgctgcgtctcggctccttcacaggtttgacaacgcggatgggcgtggcggcgatggctttcatttggatctactccctggcattcacactcgcgcctctccttgggtggaatcactacataccggaaggtttggccacttggtgtagtatcgactggctttccgatgaaacatccgataagtcctacgtgtttgctattttcatcttctgctttcttgtccctgttctcattatcgtcgtgtcatacgggctgatctacgataaggttcggaaggtagcgaaaacaggcggttctgttgccaaagcagaacgcgaagtactgcgcatgacgctgctgatggttagcctgttcatgcttgcctggagtccctacgctgttatctgcatgctcgctagcttcggccctaaagacctgctccatccagtggccacagtgattcctgccatgtttgcaaagtcttctacgatgtataaccctcttatctatgtgttcatgaacaaacaattcaggcggagccttaaagttcttctcggaatgggagtagaggacctgaactccgagagtgagagagcaactggtggtacagccacgaatcaagtcgccgcgacg

SEQ ID NO: 27 - protein, 631 aa, synthetic construct
  REGION 1..631: note = PdCO2-mScarlet open reading frame AA sequence
MDGENLTIPNPVTELMDTPINSTYFQNLNAETDGGNHYIYNAFTATDYNICAAYLFFIACLGVSLNVLVLVLFIKDRKLRSPNNFLYVSLALGDLLVAVFGTAFKFIITARKTLLREEDGFCKWYGFITYLGGLAALMTLSVIAFVRCLAVLRLGSFTGLTTRMGVAAMAFIWIYSLAFTLAPLLGWNHYIPEGLATWCSIDWLSDETSDKSYVFAIFIFCFLVPVLIIVVSYGLIYDKVRKVAKTGGSVAKAEREVLRMTLLMVSLFMLAWSPYAVICMLASFGPKDLLHPVATVIPAMFAKSSTMYNPLIYVFMNKQFRRSLKVLLGMGVEDLNSESERATGGTATNQVAATTETSQVAPAPRARDPTGKSRITSEGEYIPLDQIDINVVMVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYKFCYENEV

SEQ ID NO: 28 - DNA, 1900 bp, synthetic construct
  misc_feature 1..1900: note = PdCO2-mScarlet open reading frame NA sequence
  source 1..1900: mol_type = other DNA
atggacggggagaacttgacgattccaaatcctgtcaccgagttgatggataccccaatcaacagcacgtatttccaaaaccttaatgcggaaacggacggcggaaatcattacatctacaatgccttcacagccaccgattacaacatttgtgcagcttaccttttcttcattgcttgtctgggagtcagtctcaacgttttggtgttggtcctctttatcaaagacagaaagttgaggagccccaataacttcctgtatgtaagtctcgctctcggggatctgttggttgcagttttcggcaccgcatttaagttcattattacagctcgcaaaaccctgttgcgagaagaagacggtttctgcaaatggtacggcttcataacatacctcggcgggctcgcggcactgatgaccctgagcgttattgcttttgttcgttgtctggccgtgctgcgtctcggctccttcacaggtttgacaacgcggatgggcgtggcggcgatggctttcatttggatctactccctggcattcacactcgcgcctctccttgggtggaatcactacataccggaaggtttggccacttggtgtagtatcgactggctttccgatgaaacatccgataagtcctacgtgtttgctattttcatcttctgctttcttgtccctgttctcattatcgtcgtgtcatacgggctgatctacgataaggttcggaaggtagcgaaaacaggcggttctgttgccaaagcagaacgcgaagtactgcgcatgacgctgctgatggttagcctgttcatgcttgcctggagtccctacgctgttatctgcatgctcgctagcttcggccctaaagacctgctccatccagtggccacagtgattcctgccatgtttgcaaagtcttctacgatgtataaccctcttatctatgtgttcatgaacaaacaattcaggcggagccttaaagttcttctcggaatgggagtagaggacctgaactccgagagtgagagagcaactggtggtacagccacgaatcaagtcgccgcgacgaccgagacttcccaggttgctcctgctccgcgagcccgagatccaaccggtaagagcaggatcaccagcgagggcgagtacatccccctggaccagatcgacatcaacgtggtgatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagttctgctacgagaacgaggtgtaactag

SEQ ID NO: 29 - protein, 343 aa, synthetic construct
  REGION 1..343: note = LcPP aa sequence
MENLTSLDLLPNGEVPLMPRYGFTILAVIMAVFTIASLVLNSTVVIVTLRHRQLRHPLNFSLVNLAVADLGVTVFGASLVVETNAVGYFNLGRVGCVIEGFAVAFFGIAALCTIAVIAVDRFVVVCKPLGTLMFTRRHALLGIAWAWLWSFVWNTPPLFGWGSYELEGVRTSCAPDWYSRDPANVSYITSYFAFCFAIPFLVIVVAYGRLMWTLHQVAKLGMGESGSTAKAEAQVSRMVVVMVVAFLVCWLPYALFAMIVVTKPDVYIDPVIATLPMYLTKTSTVYNPIIYIFMNRQFRDCAVPFLLCGRNPWAEPSSESATAASTSATSVTLASAPGQVSPS

SEQ ID NO: 30 - DNA, 1029 bp, synthetic construct
  misc_feature 1..1029: note = LcPP na sequence
  source 1..1029: mol_type = other DNA
atggaaaatctgacttccctggatctgctgcccaacggagaggtcccactgatgccccggtacggctttactattctggctgtgattatggccgtgttcaccatcgcaagtctggtcctgaactcaactgtggtcattgtgaccctgcgacaccgacagctgaggcatcctctgaacttttccctggtgaatctggctgtcgcagacctgggcgtgacagtcttcggagcttctctggtggtcgagactaacgcagtggggtactttaatctgggacgcgtggggtgcgtcatcgaagggttcgccgtggctttctttggcattgccgctctgtgcaccatcgctgtgattgcagtcgatcgatttgtggtggtgtgcaagcccctgggaaccctgatgttcacaaggagacacgcactgctgggaatcgcatgggcatggctgtggagcttcgtgtggaacacaccccctctgttcggctggggaagctacgagctggaaggagtgagaactagctgcgctcctgactggtattcccgggaccccgccaacgtgagctacatcacatcttatttcgcattttgtttcgccatccccttcctggtcatcgtcgtggcttacggccggctgatgtggactctgcatcaggtggccaagctggggatgggcgagtctggaagtaccgctaaagcagaagcccaggtgagtcgcatggtcgtggtcatggtggtcgcctttctggtctgttggctgccctatgccctgttcgctatgatcgtggtcaccaagcctgacgtgtacatcgatccagtcattgccacactgcccatgtatctgaccaaaacaagcaccgtgtacaaccccatcatctacatcttcatgaatcgacagttcagggactgcgccgtgcctttcctgctgtgcggcaggaatccctgggcagagcccagctccgaatctgccacagcagcctcaaccagcgccacaagtgtgactctggcttcagcaccaggacaggtctccccatcc

SEQ ID NO: 31 - protein, 620 aa, synthetic construct
  REGION 1..620: note = LcPP -mScarlet open reading frame AA sequence
MENLTSLDLLPNGEVPLMPRYGFTILAVIMAVFTIASLVLNSTVVIVTLRHRQLRHPLNFSLVNLAVADLGVTVFGASLVVETNAVGYFNLGRVGCVIEGFAVAFFGIAALCTIAVIAVDRFVVVCKPLGTLMFTRRHALLGIAWAWLWSFVWNTPPLFGWGSYELEGVRTSCAPDWYSRDPANVSYITSYFAFCFAIPFLVIVVAYGRLMWTLHQVAKLGMGESGSTAKAEAQVSRMVVVMVVAFLVCWLPYALFAMIVVTKPDVYIDPVIATLPMYLTKTSTVYNPIIYIFMNRQFRDCAVPFLLCGRNPWAEPSSESATAASTSATSVTLASAPGQVSPSTETSQVAPAPRARDPTGKSRITSEGEYIPLDQIDINVVMVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYKFCYENEV

SEQ ID NO: 32 - DNA, 1863 bp, synthetic construct
  misc_feature 1..1863: note = LcPP -mScarlet open reading frame NA sequence
  source 1..1863: mol_type = other DNA
atggaaaatctgacttccctggatctgctgcccaacggagaggtcccactgatgccccggtacggctttactattctggctgtgattatggccgtgttcaccatcgcaagtctggtcctgaactcaactgtggtcattgtgaccctgcgacaccgacagctgaggcatcctctgaacttttccctggtgaatctggctgtcgcagacctgggcgtgacagtcttcggagcttctctggtggtcgagactaacgcagtggggtactttaatctgggacgcgtggggtgcgtcatcgaagggttcgccgtggctttctttggcattgccgctctgtgcaccatcgctgtgattgcagtcgatcgatttgtggtggtgtgcaagcccctgggaaccctgatgttcacaaggagacacgcactgctgggaatcgcatgggcatggctgtggagcttcgtgtggaacacaccccctctgttcggctggggaagctacgagctggaaggagtgagaactagctgcgctcctgactggtattcccgggaccccgccaacgtgagctacatcacatcttatttcgcattttgtttcgccatccccttcctggtcatcgtcgtggcttacggccggctgatgtggactctgcatcaggtggccaagctggggatgggcgagtctggaagtaccgctaaagcagaagcccaggtgagtcgcatggtcgtggtcatggtggtcgcctttctggtctgttggctgccctatgccctgttcgctatgatcgtggtcaccaagcctgacgtgtacatcgatccagtcattgccacactgcccatgtatctgaccaaaacaagcaccgtgtacaaccccatcatctacatcttcatgaatcgacagttcagggactgcgccgtgcctttcctgctgtgcggcaggaatccctgggcagagcccagctccgaatctgccacagcagcctcaaccagcgccacaagtgtgactctggcttcagcaccaggacaggtctccccatccaccgagaccagccaggtggctcctgctccgcgagcccgagatccaaccggtaagagcaggatcaccagcgagggcgagtacatccccctggaccagatcgacatcaacgtggtgatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagttctgctacgagaacgaggtgtaa

SEQ ID NO: 33 - protein, 342 aa, synthetic construct
  REGION 1..342: note = medakaTMT1A aa sequence
MLVSNVSLGGCAEFNSALCAGAGEEHLGGGSYRTTLTPTGHLIVAVCLGFIGTFGLVNNLLVLVLFCRYKILRSPINLLLINISISDLLVCVLGTPFSFAASTQGRWLIGEGGCVWYGFANSLCGIVSLISLAVLSYERYSTMMTPAEADSSNYRKISLGIILSWGYSLLWTLPPLFGWSHYGPEGPGTTCSVDWTAKTANNISYIICLFVFCLIVPFMVIVFCYGKLLYAIKQVSGINVSVSRKREQRVLFMVVIMVICYLLCWLPYGIMALLATFGPPDLVTPEASIIPSVLAKTSTAINPVIYVFMNKQFFRCFQAMLRCKAPLRGSSARSSSKVATKA

SEQ ID NO: 34 - DNA, 1026 bp, synthetic construct
  misc_feature 1..1026: note = medakaTMT1A na sequence
  source 1..1026: mol_type = other DNA
atgctagtcagtaatgtaagcctgggtgggtgcgccgaattcaattccgctctttgtgcgggcgctggagaagaacacctcggcggtggttcatatcggaccacgctaaccccgactggccatctcattgtcgctgtgtgccttgggttcattggcactttcgggcttgtaaataatttgcttgtcttggtcctattctgtcgttataagattctgcggagccctatcaatcttctacttattaatatcagtatttccgacctgctcgtttgcgtgctgggcacccctttctcctttgctgctagtacacaaggcaggtggttgattggcgagggcggatgtgtatggtacggatttgcgaacagcctgtgtgggatcgttagcctaatttcccttgctgtcctttcttatgaacgttactctactatgatgacccccgcggaagccgactcaagtaattaccggaaaataagtctcggtatcatcctctcctggggctatagtttgctctggacgttgccccctttgtttggctggagtcattacgggcccgaaggaccaggaaccacctgtagcgtcgattggaccgccaagaccgccaataacattagctatattatctgcctgtttgtgttctgtcttatcgtgccgtttatggtgattgtattttgctatggtaaactgctgtacgctattaaacaagtgagcggaattaatgtcagtgtaagtaggaaacgagaacaacgcgtgctctttatggtggtcattatggtcatatgctacctgctttgttggctcccttacggcattatggcccttctcgcaacgtttggacccccagacctcgtcaccccagaagcctctatcataccctcagttctcgcgaagactagtaccgctataaatccagttatctatgtctttatgaataagcagttcttcaggtgtttccaagcaatgcttaggtgtaaagctccactgcgcgggagctcagcaaggtccagctccaaagttgctacaaaagct

SEQ ID NO: 35 - protein, 619 aa, synthetic construct
  REGION 1..619: note = medakaTMT1A -mScarlet open reading frame AA sequence
MLVSNVSLGGCAEFNSALCAGAGEEHLGGGSYRTTLTPTGHLIVAVCLGFIGTFGLVNNLLVLVLFCRYKILRSPINLLLINISISDLLVCVLGTPFSFAASTQGRWLIGEGGCVWYGFANSLCGIVSLISLAVLSYERYSTMMTPAEADSSNYRKISLGIILSWGYSLLWTLPPLFGWSHYGPEGPGTTCSVDWTAKTANNISYIICLFVFCLIVPFMVIVFCYGKLLYAIKQVSGINVSVSRKREQRVLFMVVIMVICYLLCWLPYGIMALLATFGPPDLVTPEASIIPSVLAKTSTAINPVIYVFMNKQFFRCFQAMLRCKAPLRGSSARSSSKVATKATETSQVAPAPRARDPTGKSRITSEGEYIPLDQIDINVVMVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYKFCYENEV

SEQ ID NO: 36 - DNA, 1860 bp, synthetic construct
  misc_feature 1..1860: note = medakaTMT1A -mScarlet open reading frame NA sequence
  source 1..1860: mol_type = other DNA
atgctagtcagtaatgtaagcctgggtgggtgcgccgaattcaattccgctctttgtgcgggcgctggagaagaacacctcggcggtggttcatatcggaccacgctaaccccgactggccatctcattgtcgctgtgtgccttgggttcattggcactttcgggcttgtaaataatttgcttgtcttggtcctattctgtcgttataagattctgcggagccctatcaatcttctacttattaatatcagtatttccgacctgctcgtttgcgtgctgggcacccctttctcctttgctgctagtacacaaggcaggtggttgattggcgagggcggatgtgtatggtacggatttgcgaacagcctgtgtgggatcgttagcctaatttcccttgctgtcctttcttatgaacgttactctactatgatgacccccgcggaagccgactcaagtaattaccggaaaataagtctcggtatcatcctctcctggggctatagtttgctctggacgttgccccctttgtttggctggagtcattacgggcccgaaggaccaggaaccacctgtagcgtcgattggaccgccaagaccgccaataacattagctatattatctgcctgtttgtgttctgtcttatcgtgccgtttatggtgattgtattttgctatggtaaactgctgtacgctattaaacaagtgagcggaattaatgtcagtgtaagtaggaaacgagaacaacgcgtgctctttatggtggtcattatggtcatatgctacctgctttgttggctcccttacggcattatggcccttctcgcaacgtttggacccccagacctcgtcaccccagaagcctctatcataccctcagttctcgcgaagactagtaccgctataaatccagttatctatgtctttatgaataagcagttcttcaggtgtttccaagcaatgcttaggtgtaaagctccactgcgcgggagctcagcaaggtccagctccaaagttgctacaaaagctacagagacaagccaagtggcgcctgctccgcgagcccgagatccaaccggtaagagcaggatcaccagcgagggcgagtacatccccctggaccagatcgacatcaacgtggtgatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagttctgctacgagaacgaggtgtaa

SEQ ID NO: 37 - protein, 341 aa, synthetic construct
  REGION 1..341: note = zPP1 aa sequence
MHEEMESETSTAASGSIAEVMPRTGYTILAVIIGVFSVCGVILNVTVITVTLKYKQLRQPLNFALVNLAVADLGCAVFGGLPTVVTNAMGYFSLGRVGCVLEGFAVAFFGIAALCSVAVIALERCMVVCRPVGSISFQTRHAVFGVAVSWVWSFIWNTPPLFGWGRFELEGVRTSCAPDWYSRDLANVSFIVCYFLLCFALPFSVIVYSYTRLLWTLRQVSRLQVCEGGSAARAEAQVSCMVVVMILAFLLTWLPYASFALCVILIPELYIDPVIATVPMYLTKSSTVFNPIIYIFMNRQFRDRALPFLLCGRNPWAAEAEEEEEETTVSSVSRSTSVSPA

SEQ ID NO: 38 - DNA, 1023 bp, synthetic construct
  misc_feature 1..1023: note = zPP1 na sequence
  source 1..1023: mol_type = other DNA
atgcacgaggaaatggaatcagaaacatctacagcggcctctgggagcattgctgaggtgatgccccgcactggatataccatacttgcagtgattatcggggtcttttcagtctgcggtgtaatactgaacgtaaccgttattaccgtcacacttaaatacaaacagttgcgtcaacctctgaattttgcgctcgttaatctggccgtagcagacctgggatgtgcggtctttggtggccttccaacggttgtcacaaatgccatgggatatttctctttgggccgcgtcggctgcgtactcgaaggattcgctgttgctttcttcggtatcgctgcattgtgcagcgtcgccgtaattgctctcgaacggtgcatggtggtctgcagacccgtggggtcaatcagtttccaaaccagacatgccgtgtttggagttgccgtgagctgggtatggtcatttatctggaacacaccccctctcttcggttggggcaggtttgaacttgagggagtgcggacgagttgcgctccagattggtacagtagagatcttgccaatgtcagctttatagtttgttacttcttgctgtgttttgccttgcctttctctgtgattgtatacagctacactcgtttgctctggacactccgacaagtatctcggctgcaagtatgtgaaggcggttctgcggcacgcgctgaagctcaagtatcctgcatggtagtggtcatgatactcgcttttctcctcacttggctgccatatgcaagtttcgctttgtgtgttatcctcatacctgagctctatatcgaccctgtcatcgccacggtcccaatgtatctcacaaaatcctcaactgtcttcaaccctatcatctatatatttatgaatcggcagtttagggaccgtgctctcccgtttctgctgtgcgggagaaatccatgggctgcggaagccgaggaagaagaagaggaaacaaccgtgtcaagcgtcagcaggtccactagcgtaagccccgcg

SEQ ID NO: 39 - protein, 618 aa, synthetic construct
  REGION 1..618: note = zPP1 -mScarlet open reading frame AA sequence
MHEEMESETSTAASGSIAEVMPRTGYTILAVIIGVFSVCGVILNVTVITVTLKYKQLRQPLNFALVNLAVADLGCAVFGGLPTVVTNAMGYFSLGRVGCVLEGFAVAFFGIAALCSVAVIALERCMVVCRPVGSISFQTRHAVFGVAVSWVWSFIWNTPPLFGWGRFELEGVRTSCAPDWYSRDLANVSFIVCYFLLCFALPFSVIVYSYTRLLWTLRQVSRLQVCEGGSAARAEAQVSCMVVVMILAFLLTWLPYASFALCVILIPELYIDPVIATVPMYLTKSSTVFNPIIYIFMNRQFRDRALPFLLCGRNPWAAEAEEEEEETTVSSVSRSTSVSPATETSQVAPAPRARDPTGKSRITSEGEYIPLDQIDINVVMVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYKFCYENEV

SEQ ID NO: 40 - DNA, 1857 bp, synthetic construct
  misc_feature 1..1857: note = zPP1 -mScarlet open reading frame NA sequence
  source 1..1857: mol_type = other DNA
atgcacgaggaaatggaatcagaaacatctacagcggcctctgggagcattgctgaggtgatgccccgcactggatataccatacttgcagtgattatcggggtcttttcagtctgcggtgtaatactgaacgtaaccgttattaccgtcacacttaaatacaaacagttgcgtcaacctctgaattttgcgctcgttaatctggccgtagcagacctgggatgtgcggtctttggtggccttccaacggttgtcacaaatgccatgggatatttctctttgggccgcgtcggctgcgtactcgaaggattcgctgttgctttcttcggtatcgctgcattgtgcagcgtcgccgtaattgctctcgaacggtgcatggtggtctgcagacccgtggggtcaatcagtttccaaaccagacatgccgtgtttggagttgccgtgagctgggtatggtcatttatctggaacacaccccctctcttcggttggggcaggtttgaacttgagggagtgcggacgagttgcgctccagattggtacagtagagatcttgccaatgtcagctttatagtttgttacttcttgctgtgttttgccttgcctttctctgtgattgtatacagctacactcgtttgctctggacactccgacaagtatctcggctgcaagtatgtgaaggcggttctgcggcacgcgctgaagctcaagtatcctgcatggtagtggtcatgatactcgcttttctcctcacttggctgccatatgcaagtttcgctttgtgtgttatcctcatacctgagctctatatcgaccctgtcatcgccacggtcccaatgtatctcacaaaatcctcaactgtcttcaaccctatcatctatatatttatgaatcggcagtttagggaccgtgctctcccgtttctgctgtgcgggagaaatccatgggctgcggaagccgaggaagaagaagaggaaacaaccgtgtcaagcgtcagcaggtccactagcgtaagccccgcgacagaaacaagtcaagttgcgcccgccccgcgagcccgagatccaaccggtaagagcaggatcaccagcgagggcgagtacatccccctggaccagatcgacatcaacgtggtgatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagttctgctacgagaacgaggtgtaa

SEQ ID NO: 41 - protein, 336 aa, synthetic construct
  REGION 1..336: note = pPP2 aa sequence
MKPSAFYLNASLYLGPQGEPPLPRSGFIALSVIMALLTGPAIVLNATVIIVSLMHKQLRQPLNYALVNMAVADLGTAMTGGLLSVVNNAQGYFSLGRTGCVLEGFAVSLCGIASLCTVALIAVERMFVICKPLGQMQFQKQHALGGIALAWLWSLTWNLPPLFGWGRYELEGVGTSCAPDWHSREPQNVSYVLAYFTVCFAAPFVIILVSYSKLMWTLHKVTKMACMEGGAVAKSEMTVAYMVILMVVTFLISWLPYAGLSMLVVLSPDVKIHPLVGTVPVYLAKSSTVYNPIIYIYLNKQFRKYAVPFLLCGRELEMEDELSMTTVETSNRVSPA

SEQ ID NO: 42 - DNA, 1008 bp, synthetic construct
  misc_feature 1..1008: note = pPP2 na sequence
  source 1..1008: mol_type = other DNA
atgaagccttctgcattctacctaaatgcaagtctgtacctcgggccacagggagagcccccactgcctcggagtggctttattgctctgtctgtgatcatggctttgctcacaggtccagccatagtcttgaacgctacagtgataatcgtgtccctcatgcataaacaactaaggcagcctttgaattatgcgctcgttaatatggcagtggccgatcttggtacagctatgacgggcggcctgctgtctgtggtgaataatgcgcaaggatactttagcctgggcagaacaggttgcgtactggaaggttttgctgttagtctctgcggcattgcatctctgtgtacggtggcactgatcgccgtcgaacgcatgttcgtcatttgtaaacctctgggccaaatgcaattccaaaagcagcacgcattgggcggcatcgccctggcttggctttggtctctgacatggaatctcccacccctctttgggtggggcagatatgaacttgagggtgtgggcacctcatgcgcgcccgactggcatagccgggagccacaaaatgtatcatacgtccttgcttatttcactgtgtgttttgccgcaccatttgttatcattctggtctcatatagcaaactgatgtggactctgcacaaagtcactaaaatggcttgtatggaaggtggagcagtcgcaaagtctgaaatgaccgtggcctacatggttatcctcatggtagttacattcttgatcagctggctcccctacgccgggctcagcatgctcgtggttctcagcccggatgtgaagatacacccgcttgtgggtactgttcctgtgtacctggctaaatcctctacagtatacaatcccatcatttacatttatttgaacaaacaattccgcaagtacgccgtcccatttctgctctgcggccgggaactcgaaatggaagacgagctttccatgactacagtagaaactagcaatagagttagccctgcc

SEQ ID NO: 43 - protein, 613 aa, synthetic construct
  REGION 1..613: note = pPP2 -mScarlet open reading frame AA sequence
MKPSAFYLNASLYLGPQGEPPLPRSGFIALSVIMALLTGPAIVLNATVIIVSLMHKQLRQPLNYALVNMAVADLGTAMTGGLLSVVNNAQGYFSLGRTGCVLEGFAVSLCGIASLCTVALIAVERMFVICKPLGQMQFQKQHALGGIALAWLWSLTWNLPPLFGWGRYELEGVGTSCAPDWHSREPQNVSYVLAYFTVCFAAPFVIILVSYSKLMWTLHKVTKMACMEGGAVAKSEMTVAYMVILMVVTFLISWLPYAGLSMLVVLSPDVKIHPLVGTVPVYLAKSSTVYNPIIYIYLNKQFRKYAVPFLLCGRELEMEDELSMTTVETSNRVSPATETSQVAPAPRARDPTGKSRITSEGEYIPLDQIDINVVMVSKGEAVIKEFMRFKVHMEGSMNGHEFEIEGEGEGRPYEGTQTAKLKVTKGGPLPFSWDILSPQFMYGSRAFTKHPADIPDYYKQSFPEGFKWERVMNFEDGGAVTVTQDTSLEDGTLIYKVKLRGTNFPPDGPVMQKKTMGWEASTERLYPEDGVLKGDIKMALRLKDGGRYLADFKTTYKAKKPVQMPGAYNVDRKLDITSHNEDYTVVEQYERSEGRHSTGGMDELYKFCYENEV

SEQ ID NO: 44 - DNA, 1842 bp, synthetic construct
  misc_feature 1..1842: note = pPP2 -mScarlet open reading frame NA sequence
  source 1..1842: mol_type = other DNA
atgaagccttctgcattctacctaaatgcaagtctgtacctcgggccacagggagagcccccactgcctcggagtggctttattgctctgtctgtgatcatggctttgctcacaggtccagccatagtcttgaacgctacagtgataatcgtgtccctcatgcataaacaactaaggcagcctttgaattatgcgctcgttaatatggcagtggccgatcttggtacagctatgacgggcggcctgctgtctgtggtgaataatgcgcaaggatactttagcctgggcagaacaggttgcgtactggaaggttttgctgttagtctctgcggcattgcatctctgtgtacggtggcactgatcgccgtcgaacgcatgttcgtcatttgtaaacctctgggccaaatgcaattccaaaagcagcacgcattgggcggcatcgccctggcttggctttggtctctgacatggaatctcccacccctctttgggtggggcagatatgaacttgagggtgtgggcacctcatgcgcgcccgactggcatagccgggagccacaaaatgtatcatacgtccttgcttatttcactgtgtgttttgccgcaccatttgttatcattctggtctcatatagcaaactgatgtggactctgcacaaagtcactaaaatggcttgtatggaaggtggagcagtcgcaaagtctgaaatgaccgtggcctacatggttatcctcatggtagttacattcttgatcagctggctcccctacgccgggctcagcatgctcgtggttctcagcccggatgtgaagatacacccgcttgtgggtactgttcctgtgtacctggctaaatcctctacagtatacaatcccatcatttacatttatttgaacaaacaattccgcaagtacgccgtcccatttctgctctgcggccgggaactcgaaatggaagacgagctttccatgactacagtagaaactagcaatagagttagccctgccacagaaactagtcaagtagcccctgctccgcgagcccgagatccaaccggtaagagcaggatcaccagcgagggcgagtacatccccctggaccagatcgacatcaacgtggtgatggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcaccaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggacgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaagttctgctacgagaacgaggtgtaa

(19) United States
(12) Patent Application Publication
(10) Pub. No.: US 2026/0274908 A1
(43) Pub. Date: Sep. 17, 2026

(54) BISTABLE TYPE II OPSINS AND USES THEREOF

(71) Applicant: Yeda Research and Development Co. Ltd., Rehovot (IL)

(72) Inventors: Ofer YIZHAR, Rehovot (IL); Mathias MAHN, Rehovot (IL)

(73) Assignee: Yeda Research and Development Co. Ltd., Rehovot (IL)

(21) Appl. No.: 19/570,273

(22) Filed: Mar. 18, 2026

Related U.S. Application Data
(62) Division of application No. 17/478,926, filed on Sep. 19, 2021, now Pat. No. 12,612,438.
Application No. 17/478,926 is a continuation of application No. PCT/IL2020/050330, filed on Mar. 19, 2020.

(30) Foreign Application Priority Data
Mar. 19, 2019 (IL) 265486

(51) Int. Cl. C07K 14/47; A61K 38/00; A61N 5/06; A61P 25/00

(52) U.S. Cl. CPC C07K 14/47; A61N 5/06; A61P 25/00; A61K 38/00; A61N 2005/0658

(57) ABSTRACT

Bistable type II opsins are provided. Accordingly, there is provided a polypeptide comprising a bistable type II opsin and a heterologous ER export signal and/or membrane trafficking signal. Also provided are polynucleotides encoding same, cells expressing same and methods of use thereof.

### RELATED APPLICATIONS

This application is a Division of U.S. patent application Ser. No. 17/478,926 filed on Sep. 19, 2021, which is a Continuation of PCT Patent Application No. PCT/IL2020/050330 having International Filing Date of Mar. 19, 2020, which claims the benefit of priority of Israel Patent Application No. 265486 filed on Mar. 19, 2019. The contents of the above applications are all incorporated by reference as if fully set forth herein in their entirety.

### SEQUENCE LISTING STATEMENT

The XML file, entitled 107014ReplacementSequenceListing.xml, created on May 19, 2026, comprising 81,188 bytes, submitted concurrently with the filing of this application is incorporated herein by reference.

### FIELD AND BACKGROUND OF THE INVENTION

The present invention, in some embodiments thereof, relates to bistable type II opsins and uses thereof.

Several neurological disorders and diseases associated with exocrine cells such as pancreatic and adrenal cells result from over excitation or secretion or loss of excitation or secretion.

For example, several neurological disorders, such as Parkinson's disease, dystonia, essential tremor and epilepsy are thought to result from altered patterns of neural activity in defined projection pathways in the brain. Similarly, psychiatric disorders such as depression, obsessive-compulsive disorder and addiction have been proposed to result from similar mechanisms, albeit in different brain circuits.

Deep-brain stimulation (DBS) has been shown to be effective for Parkinson's disease and several other movement disorders [e.g. Benabid A L et al. Lancet. 1991 Feb. 16; 337 (8738): 403-6; and Müller U J et al. Ann N Y Acad Sci. 2013 April; 1282:119-28]. However, the specificity of electrical DBS is limited by the non-specific effects of the stimulation currents on diverse neurons located at the site of stimulation.

In recent years, the development of cellular perturbation tools based on light sensitive proteins has resulted in a technology called optogenetics, referring to the integration of genetic and optical control to achieve gain- or loss-of-function of precisely defined events within specified cells of living tissues.

Thus, for example, optogenetics neuronal targeting combined with single-photon wide-field illumination has substantiated its enormous potential in neuroscience, enabling the optical control of entire neuronal networks and unravelling their role in the control of specific behaviors. However, while optogenetics allows robust and temporally-precise excitation of long-range projecting axons (Yizhar et al., 2011), silencing such long-range connections with existing optogenetics tools has proven more difficult (Wiegert et al., 2017). This is due to an inefficacy of most optogenetic tools to suppress synaptic transmission and to paradoxical effects of others (Mahn, et al., 2016). Suppression of axonal action potentials (APs) with potassium-conducting optogenetic tools has proven ineffective for presynaptic vesicle release inhibition (Cosentino, et al., 2015; Alberio, et al., 2018; Bernal Sierra, et al., 2018; Beck, et al., 2018). Chemogenetic tools such as hM4Di can be used for silencing presynaptic release (Stachniak, et al., 2014), but suffer from slow kinetics due to the unbinding and clearance of their small-molecule ligands. Some newly-developed optogenetic tools have also been used to selectively suppress exocytosis (Liu, et al., 2019), but these tools necessitate protein turnover to reinstate synaptic transmission and are consequently intrinsically slow.

Opsins are a major class of light-sensitive proteins that can be found across all kingdoms of life and serve a diverse range of functions. Opsins can be divided into two groups, while both types are seven-transmembrane-domain proteins belonging to the G protein-coupled receptor (GPCR) superfamily, type I opsins (e.g. the microbial opsins) are ion channels or proton/ion pumps and thus are activated by light directly, while type II opsins activate G-proteins, which then activate effector enzymes that produce metabolites to e.g. open ion channels.

Current optogenetic approaches are mainly based on the light-activated microbial opsins such as channelrhodopsin, halorhodopsin, archaerhodopsin, and cruxhalorhodopsin.

Additional background art includes:
Isoldi, M. C., et al. (2005) Proceedings of the National Academy of Sciences of the United States of America 102, 1217-1221;
Koyanagi, M., et al. (2013) Proc Natl Acad Sci USA 110, 4998-5003;
Koyanagi, M., et al. (2017) Front. Ecol. Evol., 5, article 23;
Tsukamoto et al. J. Biol. Chem. (2017) doi:10.1074/jbc.M117.793539;
Eickelbeck et al. ChemBioChem (2020) 21:612-617;
Sakai K. et al. PLOS ONE (2015) 10 (10): e0141238;
Kawano-Yamashita E. et al. PLOS ONE (2015) 10 (10): e0141280;
US Patent Application Publication No. US20130347137; and
U.S. Pat. Nos. 9,505,817, 9,757,587, 8,716,447 and 9,175,095.

### SUMMARY OF THE INVENTION

According to an aspect of some embodiments of the present invention there is provided a polypeptide comprising a bistable type II opsin and a heterologous ER export signal and/or membrane trafficking signal.

According to some embodiments of the invention, the bistable type II opsin is selected from the group consisting of OPN3, OPN4, OPN5, parapinopsin, PdCO, TMT and peropsin.

According to some embodiments of the invention, the bistable type II opsin is selected from the group consisting of OPN3, OPN4, OPN5, LcPP, DrPP2, TrPP2, PdCO and peropsin.

According to some embodiments of the invention, the bistable type II opsin is selected from the group consisting of OPN3, parapinopsin, PdCO and TMT.

According to some embodiments of the invention, the bistable type II opsin is OPN3.

According to some embodiments of the invention, the bistable type II opsin is selected form the group consisting of pufferfish teleost multiple tissue opsin (PufTMT) and mosquito OPN3 (MosOpn3).

According to some embodiments of the invention, the OPN3 is mosquito OPN3 (MosOpn3).

According to some embodiments of the invention, the parapinopsin is selected from the group consisting of Lethenteron camtschaticum parapinopsin (LcPP), zebra fish parapinopsin (zPP1) and pufferfish parapinopsin (pPP2).

According to some embodiments of the invention, the TMT is selected from the group consisting of pufferfish teleost multiple tissue opsin (PufTMT) and medaka teleost multiple tissue opsin 1A (medakaTMT1A).

According to some embodiments of the invention, the PdCO is PdCO2.

According to some embodiments of the invention, the ER export signal and/or the membrane trafficking signal is of a protein expressed in neuronal cells.

According to some embodiments of the invention, ER export signal and/or the membrane trafficking signal enables trafficking to axonal presynaptic terminals.

According to some embodiments of the invention, the ER export signal and/or the membrane trafficking signal is of a Kir2.1 polypeptide.

According to some embodiments of the invention, the ER export signal comprises SEQ ID NO: 2.

According to some embodiments of the invention, the membrane trafficking signal comprises SEQ ID NO: 1.

According to an aspect of some embodiments of the present invention there is provided a polynucleotide encoding the polypeptide.

According to some embodiments of the invention, a nucleic acid sequence encoding the bistable type II opsin is codon optimized to heterologous expression.

According to some embodiments of the invention, the codon optimized to heterologous expression is codon optimized to mammalian expression.

According to an aspect of some embodiments of the present invention there is provided a nucleic acid construct comprising the polynucleotide, and a regulatory element for directing expression of the polynucleotide in a cell.

According to an aspect of some embodiments of the present invention there is provided a cell expressing the polypeptide, the polynucleotide or the nucleic acid construct.

According to an aspect of some embodiments of the present invention there is provided a method of generating a G-protein signaling in a cell, the method comprising exposing the cell to light in a wavelength that activates the polypeptide.

According to some embodiments of the invention, the method is effected in-vitro or ex-vivo.

According to some embodiments of the invention, the method is effected in-vivo.

According to an aspect of some embodiments of the present invention there is provided a method of generating a G-protein signaling in a cell in a subject in need thereof, the method comprising:
(a) administering to the subject the polypeptide, the polynucleotide, the nucleic acid construct, or the cell; and
(b) exposing a tissue region of the subject comprising the polypeptide, the polynucleotide, the nucleic acid construct or the cell to light in a wavelength that activates the polypeptide.

According to an aspect of some embodiments of the present invention there is provided a method of treating a disease that can be alleviated by this therapy in a subject in need thereof, the method comprising:
(a) administering to the subject a therapeutically effective amount of the polypeptide, the polynucleotide, the nucleic acid construct, or the cell; and
(b) exposing a tissue region of the subject comprising the polypeptide, the polynucleotide, the nucleic acid construct or the cell to light in a wavelength that activates the polypeptide,
thereby treating the disease in the subject.

According to an aspect of some embodiments of the present invention there is provided a composition comprising the polypeptide, the polynucleotide, the nucleic acid construct, or the cell, for use in treating a disease that can be alleviated by this therapy.

According to some embodiments of the invention, the cell is a mammalian cell.

According to some embodiments of the invention, the cell is a human cell.

According to some embodiments of the invention, the cell is selected from the group consisting of a neuron, an exocrine pancreatic cell and an exocrine adrenal cell, a myocardial cell, a salivary gland cell and a lacrimal cell.

According to some embodiments of the invention, the cell is a neuron.

According to some embodiments of the invention, the disease is associated with excitability of excitable tissues.

According to some embodiments of the invention, the disease is a neurological disease.

According to some embodiments of the invention, the neurological disease is selected from the group consisting of Parkinson, pain, epilepsy, depression, essential tremor, motor neuron disease, dystonia, obsessive compulsive disorder, addiction, schizophrenia, post-traumatic stress disorder, panic disorder and anxiety.

According to some embodiments of the invention, the neurological disease is selected from the group consisting of Parkinson, pain, epilepsy, depression, essential tremor, dystonia, obsessive compulsive disorder, addiction, schizophrenia, post-traumatic stress disorder, panic disorder and anxiety.

According to some embodiments of the invention, the disease is selected from the group consisting of stress, anxiety, hypertension, hyperaldosteronism, congenital adrenal hyperplasia and hyperinsulinemia.

According to some embodiments of the invention, the G-protein signaling is a Gi/o signaling.

According to some embodiments of the invention, the wavelength is 450-650 nm.

According to some embodiments of the invention, the wavelength is 350-650 nm.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B show multiple sequence alignment of vertebrate visual and non-visual rhodopsins. FIG. 1A shows alignment of the sequences of the bovine rhodopsin (bRho), the mouse short-wavelength opsin (vSWO), the teleost multiple tissue opsin from pufferfish (PufTMT), the mosquito OPN3 opsin (MosOPN3) and the chicken OPN5 (cOPN5). FIG. 1B shows alignment of the sequences of the bovine rhodopsin (bRho), the mouse short-wavelength opsin (vSWO), the teleost multiple tissue opsin from pufferfish (PufTMT), the mosquito OPN3 opsin (MosOPN3), the chicken OPN5 (cOPN5), the Platynereis dumerilii ciliary opsin 2 (PdCO2), the medaka teleost multi-tissue opsin 1a (medakaTMT1a), the Lethenteron camtschaticum parapinopsin (LcPP), the zebrafish parapinopsin-1 (zPP1), and the pufferfish parapinopsin-2 (pPP2), Intracellular domains are labeled with a green background, extracellular domains are labeled with blue background and the transmembrane domains are in gray.

FIG. 2 is a schematic presentation of the rhodopsin proteins tested in this study. The engineered DREADD actuator hM4d, and several naturally occurring rhodopsins and engineered M4-rhodopsin chimeras, consisting of the extracellular and transmembrane domains of the rhodopsins shown on the left, each modified to contain the intracellular domains of the M4 muscarinic acetylcholine receptor (identical to the intracellular domain of hM4d).

FIGS. 3A-C demonstrate exogenous expression of the indicated bistable type II opsins in cultured mammalian neurons. FIG. 3A shows representative confocal images of neurons co-transfected with expression vectors for EYFP and the indicated rhodopsin variants. Images show fluorescence in the EYFP channel (left), the mScarlet channel (center) and the merged images (right). Scale bar, 12 μm. FIG. 3B is a graph demonstrating expression level of each one of the rhodopsin-mScarlet constructs displayed in FIG. 3A, quantified as the average pixel intensity in n=13 neurons for each construct. FIG. 3C shows high-magnification maximum projection confocal images of cultured hippocampal neurons co-expressing OPN3 (left), OPN3-M4 (middle) or eOPN3 (right) and EYFP. Bottom plots depict representative line-scans through a single confocal slice from the same neurons, demonstrating the intracellular and membrane-localized fluorescence in these three constructs. Note that eOPN3 shows enhanced membrane localization compared with both OPN3 and OPN3-M4.

FIGS. 4A-C demonstrate light-triggered G protein-coupled inwardly-rectifying potassium channel (GIRK) currents in neurons co-expressing eOPN3 and GIRK2.1 as demonstration of the light gated activation of Gi/o signaling using bistable rhodopsins. FIG. 4A shows representative current recordings from a hippocampal neuron expressing eOPN3 and GIRK2.1, following illumination with a 500 ms pulse of blue, green or red light (470 nm, 560 nm and 630 nm, respectively, marked by a black arrow). FIG. 4B is a graph demonstrating the amplitude of GIRK-mediated currents evoked by light pulses at the indicated wavelengths, consistent with a green light absorption maximum of the dark-adapted state of this opsin. Individual recordings are shown as black markers, average and s.e.m. are indicated by the bar graph. FIG. 4C is a graph demonstrating time constant of GIRK current onset (τₒₙ) calculated from GIRK currents evoked with light at the indicated wavelengths. Individual recordings are shown as black markers, average and s.e.m. are indicated by the bar graph.

FIGS. 5A-B demonstrate no change in the intrinsic excitability of neurons expressing OPN3-mScarlet or eOPN3-mScarlet in the absence of light, indicative of construct expression without detrimental effects on cell health. FIG. 5A shows the intrinsic electrophysiological properties of cultured hippocampal neurons expressing OPN3-mScarlet (n=6) compared with those of neighboring untransfected neurons (n=4). FIG. 5B shows the intrinsic electrophysiological properties of cultured hippocampal neurons expressing eOPN3-mScarlet (n=8) compared with those of neighboring untransfected control neurons (n=7). These experiments demonstrate no significant change in the resting membrane potential, input resistance, action potential threshold, amplitude and half-width, and the number of action potentials evoked by increasing amounts of injected current.

FIGS. 6A-E demonstrate in-vivo reduced tone-evoked freezing during fear renewal by eOPN3-mediated inhibition of MGN and ACtx terminals in the amygdala. FIG. 6A shows a schematic diagram of auditory inputs to the amygdala. Auditory cortex (ACtx) and medial geniculate nucleus (MGN) project to the basolateral amygdala (BLA). FIG. 6B shows a schematic representation of the experimental setup. Virus encoding eOPN3-mScarlet (eOPN3, n=9) or eYFP (ctrl, n=9) was injected bilaterally into the ACtx and MGN (red structures), while optic fibers were targeted bilaterally at the amygdala (cyan). Representative images of eOPN3 expression (red) in DAPI-stained (blue) brain sections. FIG. 6C shows a schematic representation of the auditory fear conditioning paradigm. FIG. 6D demonstrates freezing scoring during the behavioral paradigm shown in FIG. 6C. Tone evoked freezing was averaged in blocks of 4 consecutive tones during the extinction and recall sessions. eOPN3 mice showed a trend to reduced freezing during the extinction session (unpaired two-sample Wilcoxon test, p=0.08). During renewal, while the amount of freezing did not differ between the two groups in the pre-tone period (unpaired two-sample Wilcoxon test, p=0.43), tone evoked freezing was lower in eOPN3 expressing mice compared to control mice (unpaired two-sample Wilcoxon test, Bonferroni corrected p=3.8.10-2). The plot depicts the median and interquartile range, single trials are depicted as small points. FIG. 6E demonstrates that tone induced freezing during the renewal session was correlated with protein expression level in the case of eOPN3, but not in control mice (ctrl: F(1,7)=0.54, p=0.48; eOPN3: F(1,7)=6.77, p=3.5.10-2). eOPN3 mice showing an expression level of less than 60% of the maximal observed expression (open circles, n=4) were excluded from the analysis shown in FIG. 6D.

FIG. 7A demonstrates light-triggered bistable G protein-coupled inwardly-rectifying potassium channel (GIRK) currents in neurons expressing the following opsins: PdCO2, LcPP, medakaTMT1A, zPP1 or pPP2 in combination with co-expressed GIRK2.1.

FIG. 7B demonstrates light triggered bistable inhibition of synaptic transmission in neurons transduced with PdCO2. Following 100 s in the dark, 422 nm light was applied for 100 ms followed by light application of 568 nm for 5 s. Upper traces show light mediated GIRK currents activated upon illumination with 422 nm. In the lower trace, GIRK currents were subtracted, revealing reduction and recovery of mEPSC frequency upon 422 and 568 nm illumination, respectively. The middle figure in FIG. 7B demonstrates mEPSC frequency reduction and recovery for multiple cells (colored lines). Data shows 5 s binned mEPSC frequency normalized to the mean frequency during 100 s pre-illumination. Mean frequency of all cells is shown in black. Below the traces, the bottom figure displays normalized mEPSC frequency for each cell as shown in FIG. 7B as a heat map.

FIG. 7C (top) demonstrates repetitive PdCO2 activation and inactivation and resulting mEPSC frequency for three successive trials on a single neuron. Single trials are shown in color, while mean frequency of all trials is shown in black. FIG. 7C (bottom) displays normalized mEPSC frequency for each trial as a heat map. Data shows 5 s binned mEPSC frequency normalized to the mean frequency during 100 s pre-illumination for each trial.

### DESCRIPTION OF SPECIFIC EMBODIMENTS OF THE INVENTION

The present invention, in some embodiments thereof, relates to bistable type II opsins and uses thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Opsins are light-sensitive membrane proteins that can be found across all kingdoms of life.

Whilst reducing the present invention to practice, the present inventors have now uncovered that bistable type II opsins can be expressed on the cell surface of mammalian neurons and specifically in axonal presynaptic terminals by the addition of an ER export signal and/or membrane trafficking signal.

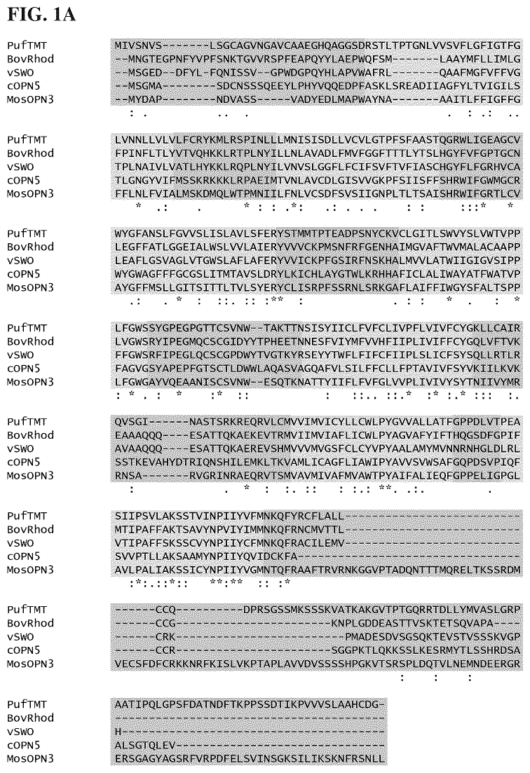

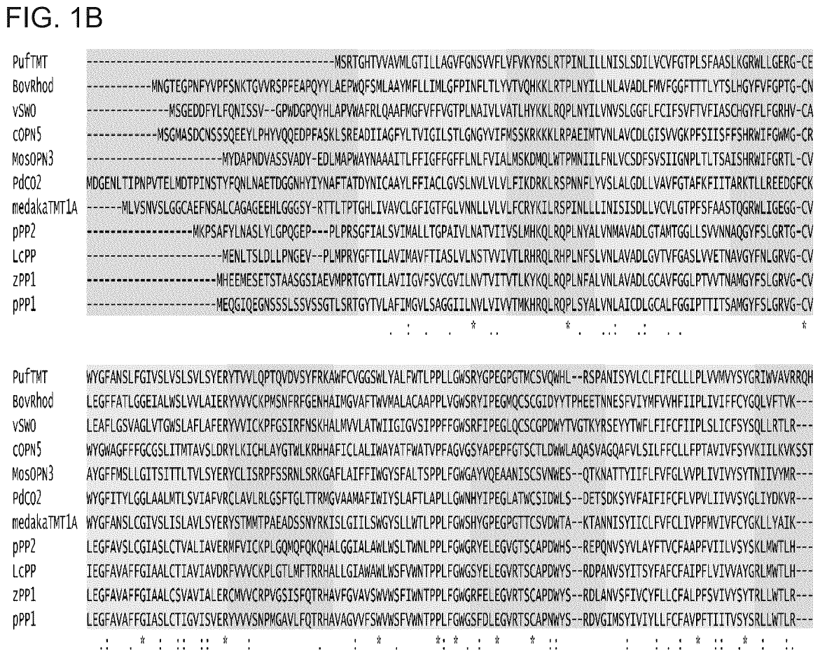

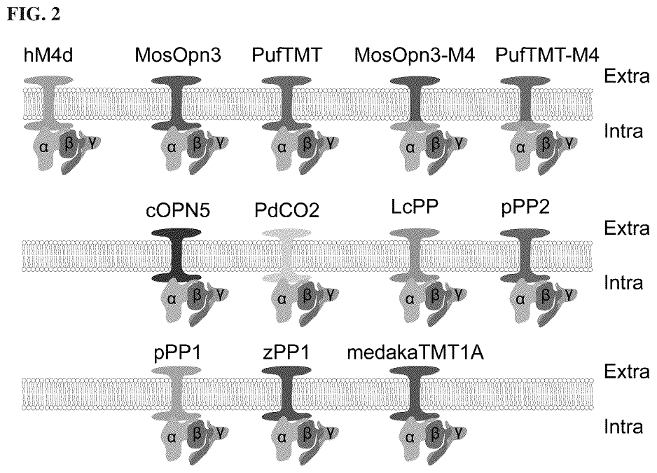

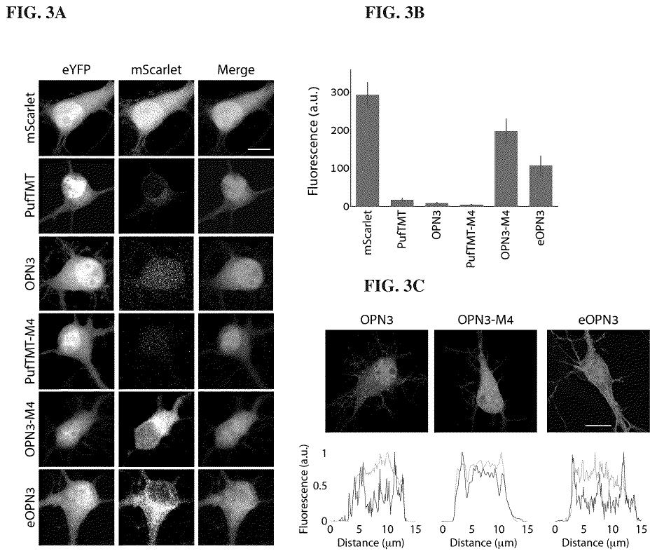

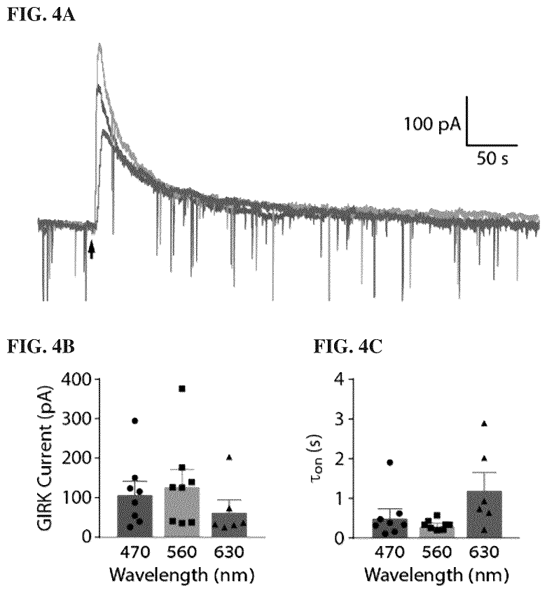

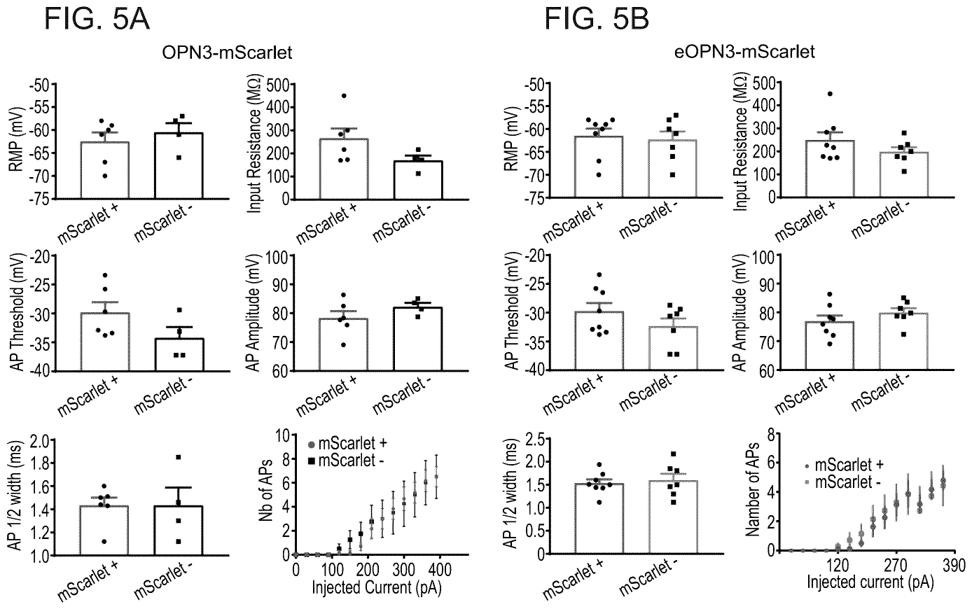

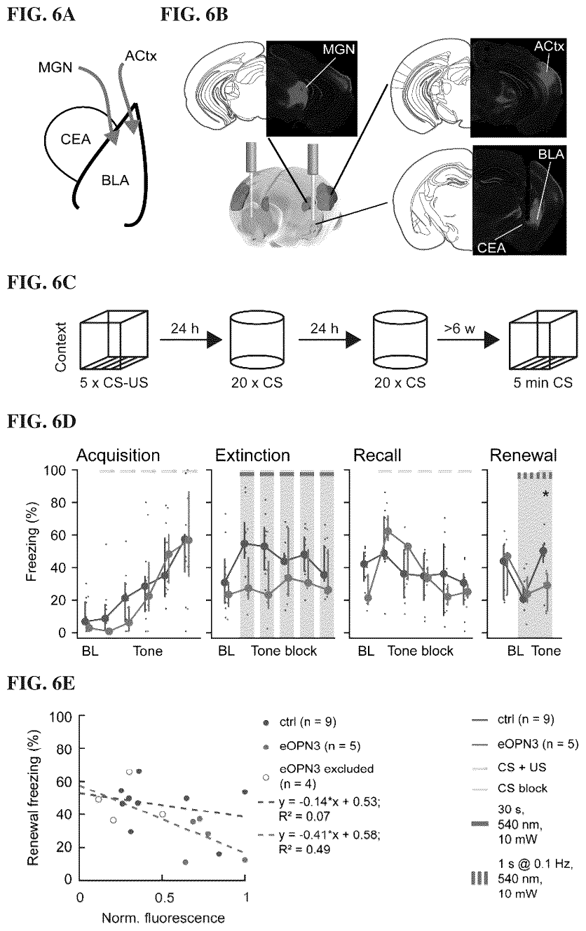

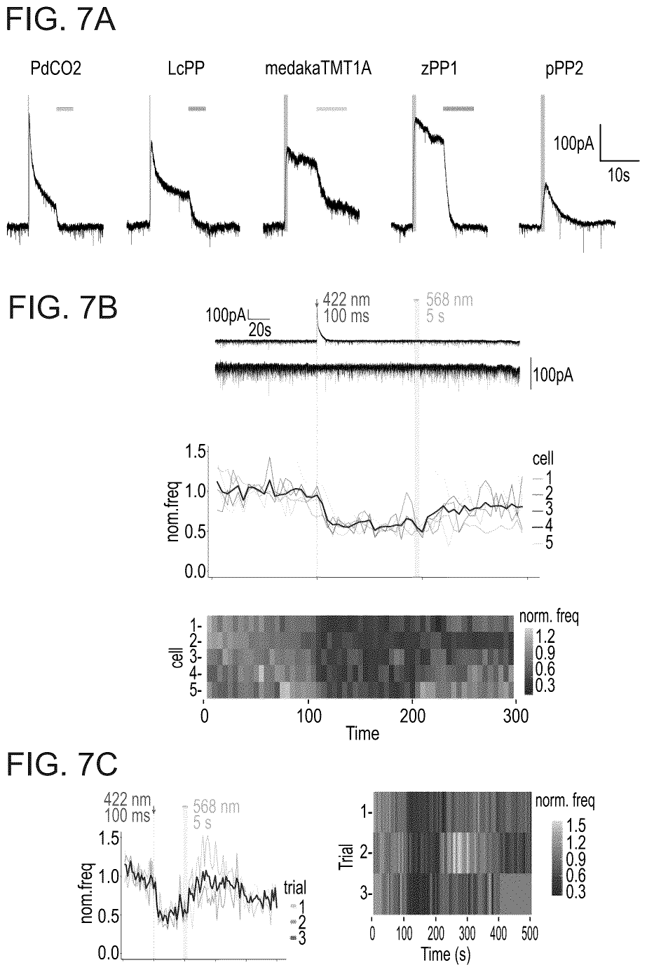

As is illustrated hereinunder and in the examples section, which follows, the present inventors show that the mosquito (Anopheles stephensi)-derived homolog of the human encephalopsin protein (OPN3) can be expressed on membranes of rat hippocampal neurons and most importantly in distal axonal presynaptic terminals by the addition of an ER export signal and membrane trafficking signal of a Kir2.1 protein (Examples 1-2, FIGS. 1A-3C). Following, the present inventors demonstrate that that the modified opsins activated the Gio pathway in neurons in response to light, leading to suppression of presynaptic release (Example 2, FIGS. 4A-5B). Furthermore, the modified mosquito OPN3 mediated in-vivo suppression of auditory afferents to the amygdala in mice, leading to impaired recall of auditory-cued fear (Example 3, FIGS. 6A-E). Furthermore, the inventors show that additional modified bistable opsins (PdCO2, LcPP, medakaTMT1A, zPP1 and pPP2) can be expressed in neurons and activate G pathway signaling in response to light and thereby reduce synaptic events (Example 4, FIGS. 7A-C). Moreover, several opsins in this group can also be switched back to the inactive state upon illumination with red-shifted wavelengths, enabling bimodal control over the opsins by light.

Consequently, the specific embodiments of the present teachings suggest polypeptides comprising a bistable type II opsin and a heterologous ER export signal and/or membrane trafficking signal, polynucleotides encoding same and method of use thereof.

Thus, according to a first aspect of the present invention, there is provided a polypeptide comprising a bistable type II opsin and a heterologous ER export signal and/or membrane trafficking signal.

A Type II opsin is a G-coupled protein receptor (GPCR) which is made light-sensitive with an attached chromophore molecule that allows it to absorb light. Most type II opsins bind 11-cis retinal as a chromophore to form a photosensitive pigment (opsin-based pigment). The isomerization of the chromophore (e.g. 11-cis to all-trans) in an opsin-based pigment upon light absorption triggers G protein activation.

According to specific embodiments, the Type II opsin activates Gi-type and Go-type G protein in a light dependent manner.

According to specific embodiments, the Type II opsin activates Gz-type G protein in a light dependent manner.

Type II opsins do not comprise an ion channel or a proton/ion pump.

As used herein, the phrase “bistable type II opsin” refers to a type II opsin which remains bound to the chromophore (e.g. retinal) following illumination (i.e. does not undergo bleaching).

Hence, a bistable type II opsin displays prolonged signal transduction following a single illumination pulse. Typically, the bistable type II opsin reverts to an original dark state through thermal relaxation after minutes in the dark or by illumination with light at a different wavelength. Methods of determining bistability of the opsin are well known in the art and include spectroscopic measurements.

According to specific embodiments, the bistable type II opsin is a naturally occurring bistable type II opsin. Such naturally occurring bistable type II opsins are known in the art and include, but are not limited to OPN3 (e.g. MosOpn3), OPN4, OPN5, parapinopsin (e.g. LcPP, zPP1, pPP2, zPP2/DrPP2, pPP2/TrPP2), PdCO (e.g. PdCO2), TMT (e.g. PufTMT, medakaTMT1A), peropsin.

According to specific embodiments, the bistable type II opsin is selected from the group consisting of OPN3, OPN4, OPN5, parapinopsin, zPP2, pPP2, PdCO, TMT and peropsin

According to specific embodiments, the bistable type II opsin is selected from the group consisting of OPN3, OPN4, OPN5, parapinopsin, PdCO and peropsin.

According to specific embodiments, the bistable type II opsin is selected from the group consisting of OPN3, parapinopsin, PdCO and TMT.

According to specific embodiments, the bistable type II opsin is selected from the group consisting of OPN3, parapinopsin and PdCO.

According to specific embodiments, the bistable type II opsin activates Gi/o signaling in a cell expressing same following exposure to light in a wavelength that activates it, as determined by e.g. GsX assay (Ballister, et al., 2018); or the ability to evoke G protein-coupled inwardly-rectifying potassium channel-mediated (GIRK) currents in neurons expressing a GIRK2-1 channel, as described in details in the Examples section which follows.

According to specific embodiments, the bistable type II opsin activates Gz signaling in a cell expressing same following exposure to light in a wavelength that activates it, as determined by e.g. GsX assay (Ballister, et al., 2018).

According to specific embodiments, the bistable type II opsin is OPN3.

As used herein, the term “OPN3” refers to the vertebrate Opsin-3, also known as encephalopsin or panopsin, and any homolog thereof.

According to specific embodiments, the OPN3 is the mosquito (Anopheles stephensi) OPN3 (MosOpn3), such as provided in the following Accession Number: BAN05625.

According to specific embodiments, the MosOpn3 amino acid sequence comprises SEQ ID NO: 8.

According to specific embodiments, the MosOpn3 amino acid sequence consists of SEQ ID NO: 8.

According to specific embodiments, the MosOpn3 amino acid sequence is the amino acid sequence described in Koyanagi et al. (Proc Natl Acad Sci USA. 2013 Mar. 26; 110 (13): 4998-5003), the content of which are fully incorporated herein by reference.

According to other specific embodiments, the MosOpn3 amino acid sequence is not the amino acid sequence described in Koyanagi et al. (Proc Natl Acad Sci USA. 2013 Mar. 26; 110 (13): 4998-5003).

According to specific embodiments, the MosOpn3 amino acid sequence comprises SEQ ID NO: 9.

According to specific embodiments, the MosOpn3 amino acid sequence consists of SEQ ID NO: 9.

According to specific embodiments, the MosOpn3 amino acid sequence does not consist of SEQ ID NO: 9.

The term “MosOpn3” also encompasses functional homologues (naturally occurring or synthetically/recombinantly produced), which exhibit the desired activity (i.e., bistable type II opsin). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID No: 8; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same.

Sequence identity or homology can be determined using any protein or nucleic acid sequence alignment algorithm such as Blast, ClustalW, and MUSCLE.

The homolog may also refer to an ortholog, a deletion, insertion, or substitution variant, including an amino acid substitution, as further described hereinbelow.

According to specific embodiments, the MosOpn3 may comprise conservative and non-conservative amino acid substitutions.

According to specific embodiments, the bistable type II opsin is TMT, also known as Teleost multiple tissue.

According to other specific embodiments, the bistable type II opsin is not TMT.

According to specific embodiments, the TMT is the pufferfish teleost multiple tissue opsin (PufTMT) such as provided in the following Accession Number: AAM90677.

According to other specific embodiments, the bistable opsin II is not the pufferfish teleost multiple tissue opsin (PufTMT).

According to specific embodiments, the PufTMT amino acid sequence comprises SEQ ID NO: 10.

According to specific embodiments, the PufTMT amino acid sequence consists of SEQ ID NO: 10.

According to specific embodiments, the TMT is TMT1A such as the medaka teleost multiple tissue opsin 1A (medakaTMT1A) such as provided in the following Accession Number: AGK24990.

According to specific embodiments, the medakaTMT1A amino acid sequence comprises SEQ ID NO: 33.

According to specific embodiments, the medakaTMT1A T amino acid sequence consists of SEQ ID NO: 33.

According to specific embodiments, the PufTMT or medakaTMT1A amino acid sequence is the amino acid sequence described in Sakai K. et al. [PLOS ONE (2015) 10 (10): e0141238], the content of which are fully incorporated herein by reference.

The terms “PufTMT”, “medakaTMT1A” also encompass functional homologues (naturally occurring or synthetically/recombinantly produced), which exhibit the desired activity (i.e., bistable type II opsin). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID No: 10, 33, respectively; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same.

According to specific embodiments, the TMT (e.g. PufTMT, medaka TMT1A) may comprise conservative and non-conservative amino acid substitutions.

According to specific embodiments, the bistable type II opsin is parapinopsin. Non-limiting examples of parapinopsins include Lethenteron camtschaticum parapinopsin (LcPP), zebrafish parapinopsin 1 (zPP1), zebrafish parapinopsin 2 [zPP2, also known as Danio rerio parapinopsin2 (drPP2)], pufferfish parapinopsin (pPP2, also known as TrPP2).

According to specific embodiments, the parapinopsin is the Lethenteron camtschaticum (Lamprey) parapinopsin (LcPP) such as provided in the following Accession Number: BAD13381.

According to specific embodiments, the LcPP amino acid sequence comprises SEQ ID NO: 29.

According to specific embodiments, the LcPP amino acid sequence consists of SEQ ID NO: 29.

According to specific embodiments, the LcPP amino acid sequence is the amino acid sequence described in Eickelbeck et al. [ChemBioChem (2020) 21:612-617], the content of which are fully incorporated herein by reference.

According to specific embodiments, the parapinopsin is the zebra fish parapinopsin 1 (zPP1) such as provided in the following Accession Number: AB626966.

According to specific embodiments, the zPP1 amino acid sequence comprises SEQ ID NO: 37.

According to specific embodiments, the zPP1 amino acid sequence consists of SEQ ID NO: 37.

According to specific embodiments, the zPP1 amino acid sequence is the amino acid sequence described in Kawano-Yamashita E. et al. [PLOS ONE (2015) 10 (10): e0141280], the content of which are fully incorporated herein by reference.

According to specific embodiments, the parapinopsin is the pufferfish parapinopsin (pPP2) such as provided in the following Accession Number: AB626965.

According to specific embodiments, the pPP2 amino acid sequence comprises SEQ ID NO: 41.

According to specific embodiments, the pPP2 amino acid sequence consists of SEQ ID NO: 41.

The terms “LcPP”, “zPP1”, “pPP2” also encompass functional homologues (naturally occurring or synthetically/recombinantly produced), which exhibit the desired activity (i.e., bistable type II opsin). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID No: 29, 37, 41, respectively; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same.

According to specific embodiments, the parapinopsin (e.g. LcPP, zPP1, pPP2) may comprise conservative and non-conservative amino acid substitutions. According to specific embodiments, the bistable type II opsin is PdCO, also known as Platynereis dumerilii ciliary opsin.

According to specific embodiments, the PdCO is the PdCO2 such as provided in the following Accession Number: AY692353.

According to specific embodiments, the PdCO2 amino acid sequence comprises SEQ ID NO: 25.

According to specific embodiments, the PdCO2 amino acid sequence consists of SEQ ID NO: 25.

According to specific embodiments, the PdCO amino acid sequence is the amino acid sequence described in Tsukamoto et al. [J. Biol. Chem. (2017) doi:10.1074/jbc.M117.793539], the content of which are fully incorporated herein by reference.

The term “PdCO2” also encompasses functional homologues (naturally occurring or synthetically/recombinantly produced), which exhibit the desired activity (i.e., bistable type II opsin). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID No: 25; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same.

According to specific embodiments, the PdCO2 may comprise conservative and non-conservative amino acid substitutions.

According to specific embodiments, the bistable type II opsin is selected from the group consisting of MosOpn3, LcPP, zPP1, pPP2, PdCO2, PufTMT and medakaTMT1A.

According to specific embodiments, the bistable type II opsin is selected from the group consisting of MosOpn3, LcPP, zPP1, pPP2, PdCO2 and medakaTMT1A.

The polypeptides disclosed herein comprises an ER export signal and/or a membrane trafficking signal heterologous to the bistable type II opsin.

As used herein, the term “heterologous” refers to a sequence which is not native to the bistable type II opsin at least in localization or is completely absent from the native sequence of the polypeptide. The heterologous moiety forms a chimeric or a fusion polypeptide.

According to specific embodiments, the heterologous ER export signal and/or membrane trafficking signal is located C-terminally to the bistable type II opsin.

According to specific embodiments, the heterologous ER export signal and/or membrane trafficking signal enables trafficking to axonal presynaptic terminals.

ER export signals are known in the art, and disclosed e.g. in Stockklausner et al., FEBS Lett.; 493 (2-3): 129-133 March 2001; Ma et al., Science Vol. 291. no. 5502:316-319, 2001); Paulhe et al., J. Biol. Chem., Vol. 279, Issue 53, 55545-55555, Dec. 31, 2004); Farhan et al., J. Cell Sci. 121:753-761, Feb. 19, 2008; the contents of each are incorporated herein by reference in their entirety.

According to specific embodiments, the ER export signal is of a protein expressed in neuronal cells.

According to specific embodiments, the ER export signal is of a protein expressed in the axons or the presynaptic terminals of neuronal cells.

Non-limiting examples ER export signals can be the signals of the inward rectifier potassium channel Kir2.1, NgCAM, VAMP2, Neurexin, Synapsin, Synaptophysin, Synaptotagmin, SynCAM, Piccolo or Basoon.

According to specific embodiments, the ER signal is of the inward rectifier potassium channel Kir2.1.

Non-limiting examples of ER export signals that can be used with specific embodiments of the invention include, FXYENE (SEQ ID NO: 11, where X is any amino acid), e.g. FCYENEV (SEQ ID NO: 2); VXXSL (SEQ ID NO: 12, where X is any amino acid), e.g. VKESL (SEQ ID NO: 13); VLGSL (SEQ ID NO: 14); NANSFCYENEVALTSK (SEQ ID NO: 15); C-terminal valine residue; and VMI.

According to specific embodiments, the ER export signal comprises SEQ ID NO: 2.

According to specific embodiments, the ER export signal consists of SEQ ID NO: 2.

According to specific embodiments, the ER export signal amino acid sequence is 5-25 amino acids in length, e.g. 5-10, 10-15, 15-20, 20-25 amino acids in length.

Membrane trafficking signals are known in the art, and include, but are not limited to membrane trafficking signals of a protein expressed on the membranes of neuronal cells.

According to specific embodiments, the membrane trafficking signal is of a protein expressed in neuronal cells.

According to specific embodiments, the membrane trafficking signal is of a protein expressed in the axons or the presynaptic terminals of neuronal cells.

Non-limiting examples of membrane trafficking signals can be the signals of the inward rectifier potassium channel Kir2.1, the hChR2, the neuronal nicotinic acetylcholine receptor, NgCAM, VAMP2, Neurexin, Synapsin, Synaptophysin, Synaptotagmin, SynCAM, Piccolo or Basoon.

According to specific embodiments, the trafficking signal is of a Kir2.1 polypeptide.

Trafficking sequences that are suitable for use with specific embodiments include, but are not limited to KSRITSEGEYIPLDQIDINV (SEQ ID NO: 1), MDYGGALSAVGRELLFVTNPVVVNGS (SEQ ID NO: 16), MAGHSNSMALFSFSLLWLCSGVLGTEF (SEQ ID NO: 17), MGLRALMLWLLAAAGLVRESLQG (SEQ ID NO: 18), MRGTPLLLVVSLFSLLQD (SEQ ID NO: 19).

According to specific embodiments, the membrane trafficking signal comprises SEQ ID NO: 1.

According to specific embodiments, the membrane trafficking signal consisting of SEQ ID NO: 1.

According to specific embodiments, the membrane trafficking signal amino acid sequence is 10-50 amino acids in length, e.g. 10-20, 20-30, 30-40, 40-50 amino acids in length.

Any of the components comprised in the polypeptide as described herein may be linked to each other directly of via a linker, each possibility represents a separate embodiment of the present invention.

Any linker known in the art can be used with specific embodiments of the invention.

According to specific embodiments, the linker may be derived from naturally-occurring multi-domain proteins or is an empirical linker as described, for example, in Chichili et al., (2013), Protein Sci. 22 (2): 153-167, Chen et al, (2013), Adv Drug Deliv Rev. 65 (10): 1357-1369, the entire contents of which are hereby incorporated by reference. In some embodiments, the linker may be designed using linker designing databases and computer programs such as those described in Chen et al., (2013), Adv Drug Deliv Rev. 65 (10): 1357-1369 and Crasto et al., (2000), Protein Eng. 13 (5): 309-312, the entire contents of which are hereby incorporated by reference.

According to specific embodiments, the linker is a synthetic linker such as PEG.

According to specific embodiments, the linker is a polypeptide.

According to specific embodiments, the linker is selected from the group consisting of PRARDP (SEQ ID NO: 4), (Gly)ₙ (where n indicates variable copy numbers), (GnSn)ₙ (where n indicates variable copy numbers), ((GₙSₙ)ₙPₙ)ₙ (where n indicates variable copy numbers) and (EAAAK)ₙ (where n indicates variable copy numbers, SEQ ID NO: 24).

The term “polypeptide” or “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

According to specific embodiments, the polypeptide may comprise epitope tags, fluorescent proteins, peptides that provide for ease of purification; cleavable linker peptides; a cell penetrating moiety, targeting moieties and the like.

According to specific embodiments, the polypeptide comprises an amino acid sequence for directing the polypeptide to a specific membrane location e.g. the axon or the presynaptic terminal.

According to specific embodiments, the polypeptide comprises a targeting moiety for directing the polypeptide to a specific cell type e.g. neuron.

According to specific embodiments, the polypeptide comprises a cell penetrating moiety, as further described hereinbelow.

Peptide bonds (—CO—NH—) within the peptide may be substituted, for example, by N-methylated amide bonds (—N(CH3)-CO—), ester bonds (—C(═O)—O—), ketomethylene bonds (—CO—CH2-), sulfinylmethylene bonds (—S(═O)—CH2-), α-aza bonds (—NH—N(R)—CO—), wherein R is any alkyl (e.g., methyl), amine bonds (—CH2-NH—), sulfide bonds (—CH2-S—), ethylene bonds (—CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2-CO—), wherein R is the “normal” side chain, naturally present on the carbon atom.

These modifications can occur at any of the bonds along the peptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The peptides of some embodiments of the invention may also include one or more modified amino acids or one or more non-amino acid monomers (e.g. fatty acids, complex carbohydrates etc).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

The peptides of some embodiments of the invention are utilized in a linear form, although it will be appreciated that in cases where cyclicization does not severely interfere with peptide characteristics, cyclic forms of the peptide can also be utilized.

Since the present peptides are preferably utilized in therapeutics or diagnostics which require the peptides to be in soluble form, the peptides of some embodiments of the invention preferably include one or more non-natural or natural polar amino acids, including but not limited to serine and threonine which are capable of increasing peptide solubility due to their hydroxyl-containing side chain.

According to specific embodiments, the peptide is provided in a formulation suitable for cell penetration that enhances intracellular delivery of the polypeptide.

For example, the polypeptide may be incorporated into a particulated delivery vehicle, e.g., a liposome, or a nano- or microparticle, by any of the methods known in the art [e.g. Liposome Technology, Vol. II, Incorporation of Drugs, Proteins, and Genetic Material, CRC Press; Monkkonen, J. et al., 1994, J. Drug Target, 2:299-308; Monkkonen, J. et al., 1993, Calcif. Tissue Int., 53:139-145; Lasic D D., Liposomes Technology Inc., Elsevier, 1993, 63-105. (chapter 3); Winterhalter M, Lasic D D, Chem Phys Lipids, 1993 September; 64 (1-3): 35-43].

Liposomes include any synthetic (i.e., not naturally occurring) structure composed of lipid bilayers, which enclose a volume. Liposomes include emulsions, foams, micelles, insoluble monolayers, liquid crystals, phospholipid dispersions, lamellar layers and the like. Liposomes can be of different sizes, may contain a low or a high pH and may be of different charge.

According to specific embodiments, cell penetrating peptides (CPP) are used to transport the polypeptide to the interior of the cells. Cell-Penetrating Peptides (CPPs) are short peptides (≤40 amino acids), with the ability to gain access to the interior of almost any cell. They are highly cationic and usually rich in arginine and lysine amino acids. They have the exceptional property of carrying into the cells a wide variety of covalently and noncovalently conjugated cargoes such as proteins, oligonucleotides, and even 200 nm liposomes. TAT (transcription activator from HIV-1), pAntp (also named penetratin, Drosophila antennapedia homeodomain transcription factor) and VP22 (from Herpes Simplex virus) are examples of CPPs that can enter cells in a non-toxic and efficient manner and may be suitable for use with some embodiments of the invention. Protocols for producing CPPs-cargos conjugates and for infecting cells with such conjugates can be found, for example L Theodore et al. [The Journal of Neuroscience, (1995) 15 (11): 7158-7167], Fawell S, et al. [Proc Natl Acad Sci USA, (1994) 91:664-668], and Jing Bian et al. [Circulation Research. (2007) 100:1626-1633].

The peptides of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final peptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the peptide compounds of some embodiments of the invention involves solid phase peptide synthesis.

Large scale peptide synthesis is described by Andersson Biopolymers 2000; 55 (3): 227-50.

According to specific embodiments, the peptide is produced by recombinant DNA technology. A “recombinant” peptide, or protein refers to a peptide, or protein produced by recombinant DNA techniques; i.e., produced from cells transformed by an exogenous DNA construct encoding the desired peptide or protein.

Thus, according to another aspect of the present invention, there is provided a polynucleotide comprising a nucleic acid sequence encoding any of the above described polypeptides.

As used herein the term “polynucleotide” refers to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

According to specific embodiments, any of the polynucleotides and nucleic acid sequences disclosed herein may comprise conservative nucleic acid substitutions. Conservatively modified polynucleotides refer to those nucleic acids which encode identical or essentially identical amino acid sequences, or where the nucleic acid does not encode an amino acid sequence, to essentially identical or associated (e.g., naturally contiguous) sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations”, which are one species of conservatively modified polynucleotides. According to specific embodiments, any polynucleotide and nucleic acid sequence described herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a polynucleotide which encodes a polypeptide is implicit in a described sequence with respect to the expression product.

According to specific embodiments, the nucleic acid sequences disclosed herein are codon optimized to heterologous (e.g. mammalian) expression.

Methods of codon optimization are known in the art and disclosed e.g. in Grote et al. (Nucleic Acid Res. Nucleic Acids Res. (2005) Jul. 1; 33 (Web Server issue): W526-W531) and include e.g. mouse codon usage optimized or human codon usage optimized versions.

Hence, according to specific embodiments, the nucleic acid sequence of the MosOpn3 comprises SEQ ID NO: 20.

According to specific embodiments, the nucleic acid sequence of the MosOpn3 consists of SEQ ID NO: 20.

According to specific embodiments, the nucleic acid sequence of the MosOpn3 comprises SEQ ID NO: 21.

According to specific embodiments, the nucleic acid sequence of the MosOpn3 consists of SEQ ID NO: 21.

According to specific embodiments, the nucleic acid sequence of the PufTMT comprises SEQ ID NO: 22.

According to specific embodiments, the nucleic acid sequence of the PufTMT consists of SEQ ID NO: 22.

According to specific embodiments, the nucleic acid sequence of the PufTMT comprises SEQ ID NO: 23.

According to specific embodiments, the nucleic acid sequence of the PufTMT consists of SEQ ID NO: 23.

According to specific embodiments, the nucleic acid sequence of the medakaTMT1A comprises SEQ ID NO: 34.

According to specific embodiments, the nucleic acid sequence of the medakaTMT1A consists of SEQ ID NO: 34.

According to specific embodiments, the nucleic acid sequence of the LcPP comprises SEQ ID NO: 30.

According to specific embodiments, the nucleic acid sequence of the LcPP consists of SEQ ID NO: 30.

According to specific embodiments, the nucleic acid sequence of the zPP1 comprises SEQ ID NO: 38.

According to specific embodiments, the nucleic acid sequence of the zPP1 consists of SEQ ID NO: 38.

According to specific embodiments, the nucleic acid sequence of the pPP2 comprises SEQ ID NO: 42.

According to specific embodiments, the nucleic acid sequence of the pPP2 consists of SEQ ID NO: 42.

According to specific embodiments, the nucleic acid sequence of the PdCO2 comprises SEQ ID NO: 26.

According to specific embodiments, the nucleic acid sequence of the PdCO2 consists of SEQ ID NO: 26.

To express an exogenous polypeptide in mammalian cells, a polynucleotide sequence encoding the polypeptide is preferably ligated into a nucleic acid construct suitable for mammalian cell expression. Such a nucleic acid construct includes a promoter sequence for directing transcription of the polynucleotide sequence in the cell in a constitutive or inducible manner.

Hence, according to specific embodiments, there is provided nucleic acid construct comprising the polynucleotide and a regulatory element for directing expression of said polynucleotide in a cell.

According to specific embodiments, the regulatory element is a heterologous regulatory element.

The nucleic acid construct (also referred to herein as an “expression vector”) of some embodiments of the invention includes additional sequences which render this vector suitable for replication and integration in prokaryotes, eukaryotes, or preferably both (e.g., shuttle vectors). In addition, typical cloning vectors may also contain a transcription and translation initiation sequence, transcription and translation terminator and a polyadenylation signal. By way of example, such constructs will typically include a 5′LTR, a tRNA binding site, a packaging signal, an origin of second-strand DNA synthesis, and a 3′ LTR or a portion thereof.

The nucleic acid construct of some embodiments of the invention typically includes a signal sequence for secretion of the peptide from a cell in which it is placed. Preferably the signal sequence for this purpose is a mammalian signal sequence or the signal sequence of the polypeptide variants of some embodiments of the invention.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], pancreas-specific promoters [Edlunch et al. (1985) Science 230:912-916] or mammary gland-specific promoters such as the milk whey promoter (U.S. Pat. No. 4,873,316 and European Application Publication No. 264,166).

According to specific embodiments, promoter is a neuron specific promoter. Non-limiting examples of neuron-specific promoters include the neurofilament promoter [Byrne et al. (1989) Proc. Natl. Acad. Sci. USA 86:5473-5477; or GenBank HUMNFL, L04147], neuron-specific enolase (NSE) promoter (see, e.g., EMBL HSENO2, X51956; see also, e.g., U.S. Pat. Nos. 6,649,811, 5,387,742); aromatic amino acid decarboxylase (AADC) promoter; synapsin promoter (see, e.g., GenBank HUMSYNIB, M55301); thy-1 promoter (see, e.g., Chen et al. (1987) Cell 51:7-19; and Llewellyn et al. (2010) Nat. Med. 16:1161); serotonin receptor promoter (see, e.g., GenBank S62283); tyrosine hydroxylase promoter (TH) (see, e.g., Nucl. Acids. Res. 15:2363-2384 (1987) and Neuron 6:583-594 (1991)); GnRH promoter (see, e.g., Radovick et al., Proc. Natl. Acad. Sci. USA 88:3402-3406 (1991)); L7 promoter (see, e.g., Oberdick et al., Science 248:223-226 (1990)); DNMT promoter (see, e.g., Bartge et al., Proc. Natl. Acad. Sci. USA 85:3648-3652 (1988)); enkephalin promoter (see, e.g., Comb et al., EMBO J. 17:3793-3805 (1988)); a myelin basic protein (MBP) promoter; CMV enhancer/platelet-derived growth factor-β promoter (see, e.g., Liu et al. (2004) Gene Therapy 11:52-60); motor neuron-specific gene Hb9 promoter (see, e.g., U.S. Pat. No. 7,632,679; and Lee et al. (2004) Development 131:3295-3306); and alpha subunit of Ca (2+)-calmodulin-dependent protein kinase II (CaMKIIa) promoter (see, e.g., Mayford et al. (1996) Proc. Natl. Acad. Sci. USA 93:13250).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long terminal repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference. Enhancers specific for distinct neuronal cell types that can be included in AAV expression vectors to gain specificity without a Cre-driver line have also been described in the arts and described e.g. in Hrvatin et al. (doi:www://doi(dot)org/10.1101/570895), which is incorporated herein by reference. Cell-type specific enhancers, such as described in e.g. Jüttner et al. [Nature Neuroscience volume 22, pages 1345-1356 (2019)] or Dimidschstein et al. (Nature Neuroscience volume 19, pages 1743-1749 (2016)], the contents of which are incorporated herein by reference, for expression in inhibitory interneurons.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

It will be appreciated that the individual elements comprised in the expression vector can be arranged in a variety of configurations. For example, enhancer elements, promoters and the like, and even the polynucleotide sequence(s) encoding the polypeptide can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1 (+/−), pGL3, pZeoSV2 (+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149:51-60).

Recombinant viral vectors are useful for in vivo expression of the polypeptides since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of some embodiments of the invention into stem cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14 (1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of some embodiments of the invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Other than containing the necessary elements for the transcription and translation of the inserted coding sequence, the expression construct of some embodiments of the invention can also include sequences engineered to enhance stability, production, purification, yield or toxicity of the expressed peptide. For example, the expression of a fusion protein or a cleavable fusion protein comprising the polypeptide of some embodiments of the invention and a heterologous protein can be engineered. Such a fusion protein can be designed so that the fusion protein can be readily isolated by affinity chromatography; e.g., by immobilization on a column specific for the heterologous protein. Where a cleavage site is engineered between the polypeptide and the heterologous protein, the polypeptide can be released from the chromatographic column by treatment with an appropriate enzyme or agent that disrupts the cleavage site [e.g., see Booth et al. (1988) Immunol. Lett. 19:65-70; and Gardella et al., (1990) J. Biol. Chem. 265:15854-15859].

The present invention also contemplates cells comprising the composition described herein.

Thus, according to an aspect of the present invention, there is provided a cell expressing the polypeptide, the polynucleotide encoding same or the nucleic acid construct encoding same.

As mentioned hereinabove, a variety of prokaryotic or eukaryotic cells can be used as host-expression systems to express the polypeptides of some embodiments of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the polypeptides of some embodiments of the invention.

Examples of bacterial constructs include the pET series of E. coli expression vectors [Studier et al. (1990) Methods in Enzymol. 185:60-89).

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

In cases where plant expression vectors are used, the expression of the coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hsp17.5-E or hsp17.3-B [Gurley et al. (1986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, NY, Section VIII, pp 421-463.

Other expression systems such as insects and mammalian host cell systems which are well known in the art and are further described hereinbelow can also be used by some embodiments of the invention.

According to specific embodiments, the cell is a human cell.

Suitable mammalian cells include primary cells and immortalized cell lines.

According to specific embodiment, the mammalian cell is a neuron, e.g., a non-immortalized (primary) neuron.

According to other specific embodiments, the mammalian cell is an immortalized cell line.

Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

According to specific embodiments, the cell is a cell of an excitable tissue, e.g. a neuron, an exocrine pancreatic cell, an exocrine adrenal cell, a myocardial cell, a salivary gland cell, a lacrimal cell.

According to specific embodiments, the cell is a neuronal cell or a neuronal-like cell. The cells can be of human, non-human primate, mouse, or rat origin, or derived from a mammal other than a human, non-human primate, rat, or mouse. Suitable cell lines include, but are not limited to, a human glioma cell line, e.g., SVGp12 (ATCC CRL-8621), CCF-STTG1 (ATCC CRL-1718), SW 1088 (ATCC HTB-12), SW 1783 (ATCC HTB-13), LLN-18 (ATCC CRL-2610), LNZTA3WT4 (ATCC CRL-11543), LNZTA3WT11 (ATCC CRL-11544), U-138 MG (ATCC HTB-16), U-87 MG (ATCC HTB-14), H4 (ATCC HTB-148), and LN-229 (ATCC CRL-2611); a human medulloblastoma-derived cell line, e.g., D342 Med (ATCC HTB-187), Daoy (ATCC HTB-186), D283 Med (ATCC HTB-185); a human tumor-derived neuronal-like cell, e.g., PFSK-1 (ATCC CRL-2060), SK-N-DZ (ATCCCRL-2149), SK-N-AS (ATCC CRL-2137), SK-N-FI (ATCC CRL-2142), IMR-32 (ATCC CCL-127), etc.; a mouse neuronal cell line, e.g., BC3H1 (ATCC CRL-1443), EOC1 (ATCC CRL-2467), C8-D30 (ATCC CRL-2534), C8-S(ATCC CRL-2535), Neuro-2a (ATCC CCL-131), NB41A3 (ATCC CCL-147), SW10 (ATCC CRL-2766), NG108-15 (ATCC HB-12317); a rat neuronal cell line, e.g., PC-12 (ATCC CRL-1721), CTX TNA2 (ATCC CRL-2006), C6 (ATCC CCL-107), F98 (ATCC CRL-2397), RG2 (ATCC CRL-2433), B35 (ATCC CRL-2754), R3 (ATCC CRL-2764), SCP (ATCC CRL-1700), OA1 (ATCC CRL-6538).

According to specific embodiments, the cell is not a visual cell.

According to specific embodiments, the cell is not a retinal cell.

According to specific embodiments, the cell does not express a G protein transducin (Gt).

According to specific embodiments, the cell expresses a Gi/o protein.

According to specific embodiments, recovery of the recombinant polypeptide is effected following an appropriate time in culture. The phrase “recovering the recombinant polypeptide” refers to collecting the whole fermentation medium containing the polypeptide and need not imply additional steps of separation or purification. Notwithstanding the above, polypeptides of some embodiments of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

The present invention also contemplates uses of the polypeptides, polynucleotides, nucleic acid constructs and cells disclosed herein in e.g. therapeutic, research and diagnostic applications.

Thus, according to an aspect of the present invention, there is provided a method of generating a G-protein signaling in a cell, the method comprising exposing a cell expressing the polypeptide, the nucleic acid or the nucleic acid construct disclosed herein to light in a wavelength that activates the polypeptide.

According to an additional or an alternative aspect of the present invention there is provided a method of determining the effect of generating a G-protein signaling in a cell, the method comprising:
(a) exposing a cell expressing the polypeptide, the nucleic acid or the nucleic acid construct disclosed herein to light in a wavelength that activates the polypeptide; and
(b) determining a biological outcome following the exposing.

The method of some embodiments of the invention can be effected in-vitro, ex-vivo or in-vivo.

According to specific embodiments, the method is effected in-vitro or ex-vivo.

According to specific embodiments, the method is effected in-vivo.

According to an additional or an alternative aspect of the present invention, there is provided a method of generating a G-protein signaling in a cell in a subject in need thereof, the method comprising:
(a) administering to the subject the polypeptide, the polynucleotide, the nucleic acid construct or the cell; and
(b) exposing a tissue region of said subject comprising said polypeptide, said polynucleotide, said nucleic acid construct or said cell to light in a wavelength that activates said polypeptide.

According to specific embodiments, following administering the polypeptide, the polynucleotide or the nucleic acid construct is expressed in a cell(s) of the subject.

According to specific embodiments, the G-protein signaling is a Gi/o signaling (e.g. inhibits the production of cAMP from ATP through the Ga subunit and/or inhibits activity of calcium channels and/or SNARE proteins through the Gβγ subunit in e.g. neuron).

According to specific embodiments, the G-protein signaling is a Gz signaling.

According to specific embodiments, when the cell is a neuronal cell, the G-protein signaling is generated in axonal presynaptic terminals. Methods of determining changes effected in axonal presynaptic terminals such as presynaptic vesicle release are known in the art and described for example in Wiegert et al. Proc Natl Acad Sci USA. 2013 Nov. 19; 110 (47): E4510-9; and Rost et al. Nat Neurosci. 2015 December; 18 (12): 1845-1852, the contents of which are fully incorporated herein by reference.

According to specific embodiments, it is possible to reverse activation of the expressed polypeptide using light in a wavelength different than the one that activates the polypeptide, enabling easier regulation of the amount and duration of activation. Thus, according to specific embodiments, the method comprising exposing the cell expressing the polypeptide, the nucleic acid or the nucleic acid construct disclosed, or a tissue region comprising the polypeptide, the nucleic acid, the nucleic acid construct or the cell disclosed herein, to light in a wavelength that inhibits activation of the polypeptide.

According to an additional or an alternative aspect of the present invention, there is provided a method of treating a disease that can be alleviated by this therapy in a subject in need thereof, the method comprising:
(a) administering to the subject a therapeutically effective amount of the polypeptide, the polynucleotide, the nucleic acid construct, or the cell; and
(b) exposing a tissue region of said subject comprising said polypeptide, said polynucleotide, said nucleic acid construct or said cell to light in a wavelength that activates said polypeptide,
   thereby treating the disease in the subject.

According to an additional or an alternative aspect of the present invention, there is provided a composition comprising the polypeptide, the polynucleotide, the nucleic acid construct, or the cell, for use in treating a disease that can be alleviated by this therapy.

The term “treating” or “treatment” refers to inhibiting, preventing or arresting the development of a pathology (disease, disorder or medical condition) and/or causing the reduction, remission, or regression of a pathology or a symptom of a pathology. Those of skill in the art will understand that various methodologies and assays can be used to assess the development of a pathology, and similarly, various methodologies and assays may be used to assess the reduction, remission or regression of a pathology.

As used herein, the term “subject” includes mammals, e.g., human beings at any age and of any gender. According to specific embodiments, the term “subject” refers to a subject who suffers from the pathology (disease, disorder or medical condition). According to specific embodiments, this term encompasses individuals who are at risk to develop the pathology.

As used herein, the term “disease is associated with excitability of excitable tissues” refers to a disease or disorder that can be ameliorated by activating a G-protein signaling in excitable cells.

According to specific embodiments, the disease can benefit from activating a Gi/o signaling in excitable cells.

According to specific embodiments, the disease can benefit from activating a Gz signaling in excitable cells.

Such excitable cell and tissues include neurons, myocardial cells, glandular tissues, salivary glands and lacrimal glands.

Thus, according to specific embodiments, the disease is a neurological disease.

As used herein, the term “neurological disease” refers to a disease that can benefit from activating G-protein signaling in neuronal cells (and specifically in axonal presynaptic terminals) or in synaptic projection pathways.

According to specific embodiments, the neurological disease can benefit from suppressing presynaptic transmission of neurons.

Non-limiting examples of such neurological diseases include, Parkinson, pain (e.g. chronic pain), epilepsy, depression, essential tremor, motor neuron disease, dystonia, obsessive compulsive disorder, addiction, schizophrenia, post-traumatic stress disorder, panic disorder, anxiety disorders.

According to specific embodiments, the disease is a motor neuron disease. Non-limiting examples of motor neuron diseases include monomelic amyotrophy (MMA), progressive muscular atrophy (PMA), sporadic amyotrophic lateral sclerosis (ALS), pseudobulbar palsy primary lateral sclerosis (PLS), progressive bulbary palsy (PBP).

As secretion of glandular factors, such as hormones is in some cases effected by the excitation of secreting cells or tissues, according to other specific embodiments, the disease is associated with cells of glandular tissues such as pancreatic exocrine cells and adrenal exocrine cells.

A non-limiting example of such diseases associated with pancreatic exocrine cells is hyperinsulinemia.

Non-limiting examples of such diseases associated with adrenal exocrine cells include stress, anxiety, hypertension, hyperaldosteronism, congenital adrenal hyperplasia.

Myocardial contraction depends on the opening and closing of a complex series of ion channels in myocardial cell membranes, the end result of this depolarization-repolarization is that the contractile filaments in the cell engage, and the cell contracts.

Hence, according to specific embodiments, the disease is associated with myocardial cells such as, but not limited to cardiac arrhythmia.

According to specific embodiments, the disease is associated with lacrimal and/or salivary secreting cells such as, but not limited to Sjorgen's syndrome.

The polypeptide, polynucleotides, nucleic acid constructs and cell of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the polypeptide, polynucleotides, nucleic acid constructs and cells accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The methods of some embodiments of the present invention comprise exposing a cell or a tissue region to a light in a wavelength that activates the polypeptide.

Such a wavelength typically depends on the type of the bistable type II opsin. Determining the suitable wavelength is well within the capabilities of the skilled in the art. According to specific embodiments, the light is an ultraviolet, blue, green or red light.

According to specific embodiments, the wavelength is 350-650 nm.

According to specific embodiments, the wavelength is 450-650 nm.

According to specific embodiments, the wavelength is 450-490 nm.

According to specific embodiments, the wavelength is about 470 nm.

According to specific embodiments, the wavelength is 540-580 nm.

According to specific embodiments, the wavelength is about 560 nm.

According to specific embodiments, the wavelength is 610-650 nm.

According to specific embodiments, the wavelength is about 630 nm.

According to specific embodiments, exposing the cell or the tissue region to light is effected by light pulses that can have a duration for any of at least 1 millisecond (ms), at least 5 ms, at least 10 ms, at least 50 ms, at least 100 ms, at least 500 ms, at least 1 sec.

According to specific embodiments, exposing the cell or the tissue region to light is effected by light pulses that can have a duration for any of about 1 millisecond (ms), about 2 ms, about 3, ms, about 4, ms, about 5 ms, about 6 ms, about 7 ms, about 8 ms, about 9 ms, about 10 ms, about 15 ms, about 20 ms, about 25 ms, about 30 ms, about 35 ms, about 40 ms, about 45 ms, about 50 ms, about 60 ms, about 70 ms, about 80 ms, about 90 ms, about 100 ms, about 200 ms, about 300 ms, about 400 ms, about 500 ms, about 600 ms, about 700 ms, about 800 ms, about 900 ms, about 1 sec, about 1.25 sec, about 1.5 sec, or about 2 sec.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization-A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Animals—Animal experiments were carried out according to the guidelines stated in directive 2010/63/EU of the European Parliament on the protection of animals used for scientific purposes. Animal experiments at the Weizmann Institute were approved by the Weizmann Institute Institutional Animal Care and Use Committee (IACUC).

Molecular cloning of bistable rhodopsin constructs—The genes encoding mScarlet, and the following opsins: OPN3, PufTMT, OPN3-M4, PufTMT-M4, PdCO2, LcPP, medakaTMT1A, zPP1 and pPP2 were synthesized using the Twist gene synthesis service (Twist Bioscience, USA). All genes were subcloned into pAAV vectors under the CamKIIα promoter and in-frame with mScarlet at the C-terminus. The enhanced expression plasmids (eOPN3, PdCO2, LcPP, medakaTMT1A, zPP1 and pPP2) were generated by adding the Kir2.1 membrane trafficking signal (KSRITSEGEYIPLDQIDINV, SEQ ID NO: 1) between the opsin and the mScarlet coding sequences and the Kir2.1 ER export signal (FCYENEV, SEQ ID NO: 2) following the C-terminus of mScarlet. The sequences of the eOPN3-mScarlet, PdCO2-mScarlet, LcPP-mScarlet, medakaTMT1A-mScarlet, zPP1-mScarlet and pPP2-mScarlet open reading frames are provided in SEQ ID NOs: 5-6, 27-28, 31-32, 35-36, 39-40 and 43-44 (the mScarlet nucleic acid sequence is provided in SEQ ID NO: 7).

Production of recombinant AAV vectors—HEK293 cells were seeded at 25%-35% confluence. The cells were transfected 24 hours later with plasmids encoding AAV rep, cap of AAV1 and AAV2 and a vector plasmid for the rAAV cassette expressing the relevant DNA using the PEI method [Grimm, D., Kay, M. A. & Kleinschmidt, J. A. Mol. Ther.: J. Am. Soc. Gene Ther. 7, 839-850 (2003)]. Cells and medium were harvested 72 hours following transfection, pelleted by centrifugation (300 g), resuspended in lysis solution ([mM]: 150 NaCl, 50 Tris-HCl; pH 8.5 with NaOH) and lysed by three freeze-thaw cycles. The crude lysate was treated with 250 U benzonase (Sigma) per 1 ml of lysate at 37° C. for 1.5 hours to degrade genomic and unpackaged AAV DNA before centrifugation at 3,000 g for 15 minutes to pellet cell debris. The virus particles in the supernatant (crude virus) were purified using heparin-agarose columns, eluted with soluble heparin, washed with phosphate buffered saline (PBS) and concentrated by Amicon columns. Viral suspension was aliquoted and stored at −80° C. Viral titers were measured using real-time PCR. In experiments that compared between different constructs, viral titers were matched by dilution to the lowest concentration. AAV vectors used for neuronal culture transduction were added 4 days following cell seeding. Recordings were carried out between 4-10 days following viral transduction. The following viral vectors were used in this study: AAV2/1&2.CamKIIα.OPN3-mScarlet, AAV2/1&2.CamKIIα.eOPN3-mScarlet, AAV2/1&2.CamKIIα.mCherry.WPRE.

Primary hippocampal neuron culture-Primary cultured hippocampal neurons were prepared from male and female P0 Sprague-Dawley rat pups (Envigo). CA1 and CA3 were isolated, digested with 0.4 mg ml⁻¹ papain (Worthington), and plated onto glass coverslips pre-coated with 1:30 Matrigel (Corning). Cultured neurons were maintained in a 5% CO₂ humidified incubator with Neurobasal-A medium (Invitrogen) containing 1.25% fetal bovine serum (FBS, Biological Industries), 4% B-27 supplement (Gibco), 2 mM Glutamax (Gibco) and plated on coverslips in a 24-wells plate at a density of 65,000 cells per well. To inhibit glial overgrowth, 200 μM fluorodeoxyuridine (FUDR, Sigma) was added following 4 days of in vitro culture (DIV).

Neurons were transfected using the calcium phosphate method [Graham, F. L. & Eb, A. J. A Virology 52, 456-467 (1973)]. Briefly, the medium of primary hippocampal neurons cultured in a 24 wells plate was collected and replaced with 400 μl serum-free MEM medium (ThermoFisher scientific). 30 μl transfection mix (2 μg plasmid DNA and 250 μM CaCl₂) in HBS at pH 7.05) were added per well. Following a 1 hour incubation the cells were washed twice with MEM and the medium was changed back to the collected original medium. Cultured neurons were used between 14-21 DIV for experiments. The following plasmids were used in this study: pcDNA3.1-GIRK2.1 [Lesage F et al. FEBS Lett. (1994) Oct. 10; 353 (1): 37-42], pAAV-CamKIIα-OPN3-mScarlet, pAAV-CamKIIα-eOPN3-mScarlet, pAAV-CamKIIα-PufTMT-mScarlet, pAAV-CamKIIα-OPN3-M4-mScarlet, pAAV-CamKIIα-PufTMT-M4-mScarlet, pAAV-CamKIIα-eYFP, pAAV-CamKIIα-PdCO2-mScarlet, pAAV-CamKIIα-LcPP-mScarlet, pAAV-CamKIIα-medakaTMT1A-mScarlet, pAAV-CamKIIα-zPP1-mScarlet, pAAV-CamKIIα-pPP2-mScarlet.

Confocal imaging and quantification—Primary cultured hippocampal neurons were transfected at 5 DIV with plasmids encoding a rhodopsin protein (mScarlet, OPN3, PufTMT, OPN3-M4, PufTMT-M4, eOPN3) along with pAAV-CamKIIα-eYFP. Four days following transfection, cells were fixed and permeabilized, washed 4 times with PBS and stained for 3 min with DAPI (5 mg/ml solution diluted 1:30,000 prior to staining). Coverslips were then mounted using PVA-DABCO (Sigma) and allowed to dry. Images of mScarlet and EYFP fluorescence were acquired using a Zeiss LSM 700 confocal microscope with a 20× magnification objective. Fluorescence was quantified using ImageJ {Schindelin: 2012ir} by marking a region containing the somatic cytoplasm using the EYFP fluorescence and then measuring the average pixel intensity in the red imaging channel.

In vitro electrophysiology-Whole-cell patch clamp recordings were performed under visual control using differential interference contrast infrared (DIC-IR) illumination on an Olympus IX-71 microscope equipped with a monochrome scientific CMOS camera (Andor Neo). Borosilicate glass pipettes (Sutter Instrument BF100-58-10) with resistances ranging from 3-7 M (2 were pulled using a laser micropipette puller (Sutter Instrument Model P-2000). For hippocampal neuron cultures, electrophysiological recordings from neurons were obtained in Tyrode's medium ([mM] 150 NaCl, 4 KCl, 2 MgCl₂, 2 CaCl₂), 10 D-glucose, 10 HEPES; 320 mOsm; pH adjusted to 7.35 with NaOH). The recording chamber was perfused at 0.5 ml min-1 and maintained at 29-32° C. Pipettes were filled using a potassium gluconate-based intracellular solution ([mM] 135 K-gluconate, 4 KCl, 2 NaCl, 10 HEPES, 4 EGTA, 4 MgATP, 0.3 NaGTP; 280 mOsm kg-1; pH adjusted to 7.3 with KOH). Whole-cell voltage clamp recordings were performed using a MultiClamp 700B amplifier, filtered at 8 kHz and digitized at 20 kHz using a Digidata 1440A digitizer (Molecular Devices). Light was delivered using a Lumencor SpecraX light engine, using band-pass filters at 475/28, 542/27 and 632/22 nm (peak wavelength/bandwidth). Light power was calibrated to be identical at all three wavelengths to allow comparison of activation efficiency. Remaining photon flux differences were less than 6%.

In vivo optogenetic silencing: AAV vectors encoding eOPN3 (AAV2/1&2.CamKIIα.eOPN3-mScarlet) or eYFP (AAV2/1&2.CamKIIα.eYFP.WPRE) were bilaterally injected into the auditory cortex (AP: −2.8 mm, ML: +/−4.15 mm DV: −2.8 mm) and medial geniculate nucleus/auditory thalamus (AP: −3.2 mm, ML: +/−1.8 mm DV: −3.65 mm). Optical fibers (200 μm diameter, NA 0.5) were bilaterally implanted above the amygdala complex (AP: −1.5 mm, ML: +/−3.0 mm DV: −4.3 mm). Mice were allowed to recover for 8-10 weeks to allow for viral expression. Prior to fear conditioning, mice were habituated to optical patch cord connection for 8 minutes on two consecutive days in the box used to transfer them from home cage to behavior room. Mice in both the eOPN3 and control group were placed in the fear conditioning chamber (Med Associates) in context A, allowed 10 minutes of habituation and then presented with five pairings of the CS (50 ms-long 5 kHz tones, delivered at 10 Hz for 30 s) and US (continuous 0.5 mA constant current foot shock for 1 second delivered with a standalone aversive stimulator, ENV-414S, Med Associates Inc., St. Albans, Vermont). Each CS co-terminated with a US, with a 60 seconds interval between CS-US pairings. On day 2 and 3, mice underwent a cued recall/extinction-learning test. These experiments were conducted in context B, which differed from context A in texture, odor and ambient light. Mice were presented with 20 repetitions of the CS, separated by 60 seconds intervals. During the extinction session, light (540 nm at 10 mW) was delivered during as well as 2 seconds prior and after CS representation. Six weeks following fear conditioning, mice were re-exposed to the fear-conditioning context for 15 minutes (renewal). Starting at minute 5, light pulses (1 s, 540 nm at 10 mW at 0.1 Hz) were delivered bilaterally through the fiber implants. Starting minute 10, a CS (50 ms-long 5 kHz tones, delivered at 10 Hz for 5 mins) was presented. Movies recorded at 40 frames per second were automatically scored for freezing by a custom written Fiji script (Schindelin, et al., 2012). To measure freezing bouts, for each frame the number of pixels with altered values compared to the previous frame were quantified. These values were temporally filtered by a Gaussian filter with 3 frames standard deviation. Only changed pixels around the mouse body were considered, to discard artifacts arising from patch-cord motion. A freezing threshold was set automatically per mouse and behavioral session by detection of the minimum in the distributions for freezing and motion peaks in the pixel change histogram. A mouse was considered to be freezing if 60 consecutive values (1.5 s) were below the freezing threshold.

### Example 1

### Exogenous Expression of Bistable Type II Opsins in Neurons

Recent work has identified a family of rhodopsins that exist in a wide range of organisms that are intrinsically bistable (FIG. 1A-B, PufTMT and MosOPN3), i.e. remain bound to the retinal chromophore after illumination and display prolonged signal transduction following a single illumination pulse (Koyanagi et al., 2013).

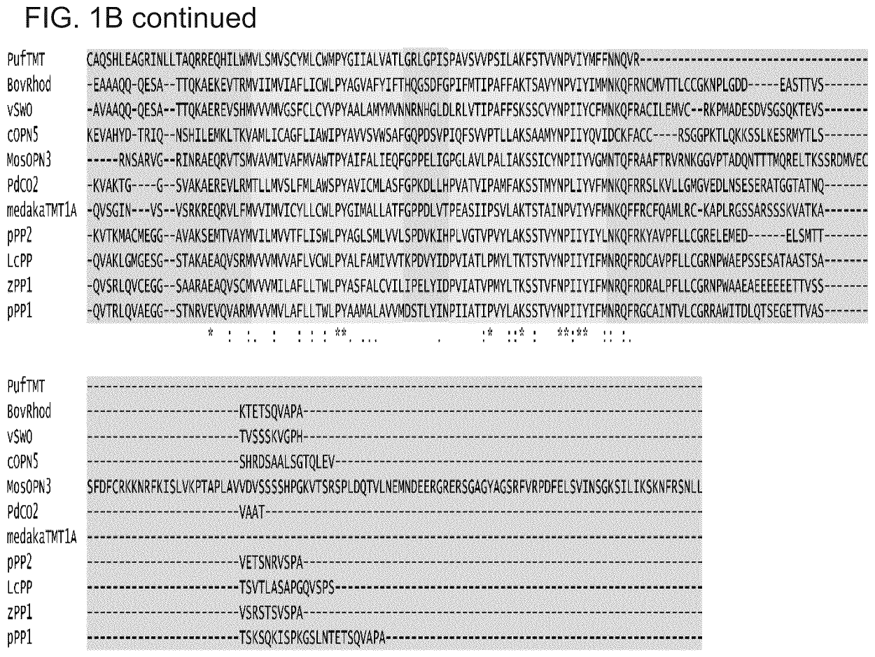

The present inventors tested several photoreceptors of this family for expression in mammalian neurons. To this end, primary cultured hippocampal neurons were transduced with mammalian codon-optimized version of PufTMT or MosOPN3. As shown in FIGS. 3A-B, the expression levels of both rhodopsins were low compared with a control vector comprising only the mScarlet fluorescent protein and mostly intracellular. In the next step, primary cultured hippocampal neurons were transduced with a chimeric photoreceptor composed of transmembrane and extracellular domains of bistable invertebrate rhodopsin and the intracellular domain of the M4 acetylcholine receptor, in order to recapitulate the M4 signaling pathway. The DREADD receptor hM4D in combination with its cognate ligand have been suggested as an attractive alternative to optogenesics for manipulation of presynaptic terminals. As shown in FIGS. 3A-B, the chimeric PufTMT-M4 did not show any detectable expression, while expression of the OPN3-M4 chimera was higher compared to the native OPN3 protein. However, expression of the OPN3-M4 was limited to the cytoplasm and almost no expression was detected on the membrane of the cells (FIG. 3C).

Following, in order to induce membrane expression of MosOPN3, primary cultured hippocampal neurons were transduced with a modified MosOPN3, referred to herein as eOPN3, which comprised an ER-export signal along with a membrane trafficking motif yielding (MosOPN3-ts-mScarlet-ER). Indeed, this modified eOPN3 was highly expressed in cultured hippocampal neurons compared with the native OPN3 and showed increased membrane targeting compared with the OPN3-M4 chimera (FIGS. 3A-C).

### Example 2

### Bistable Type II Opsins Exogenously Expressed in Neurons are Functional and Inhibit Presynaptic Transmission

To test for functional activation of the G coupled Gi/o pathway by these novel engineered bistable rhodopsins, whole-cell recordings were conducted in neurons co-transfected with plasmids encoding eOPN3 with the G protein-gated potassium channel GIRK2.1. This configuration allowed quantification of Gio pathway activity through the measurement of GIRK2-1-mediated hyperpolarizing K⁺-currents. The endogenous expression of GIRK2-1 in neuronal cell types (Lüscher & Slesinger, 2010) and its ability to form functional homotetramers (Whorton & Mackinnon, 2011) make GIRK2-1 well suited as a reporter of Gi/o pathway activation in neurons. In neurons expressing OPN3 and eOPN3 robust outward currents were recorded upon illumination with UV, blue, green and red light, with a maximum response at 560 nm (FIGS. 4A-B), consistent with previous characterization of light absorption of this rhodopsin.

Expression of exogenous proteins in mammalian neurons can lead to undesired consequences, from impaired cell health to light- or ligand-independent effects on the physiological activity or signal transduction pathways. To this end, changes in the intrinsic excitability of neurons expressing OPN3 or eOPN3 were evaluated. No significant difference between neurons expressing OPN3-mScarlet or eOPN3-mScarlet and neighboring non-expressing cells (FIGS. 5A-B). Hence, the eOPN3 variant is well-tolerated in mammalian neurons its expression does not trigger any light-independent physiological changes in neuronal excitability.

### Example 3

### Exogenous Expression of Bistable Type II Opsins Inhibits Presynaptic Transmission In-Vivo

To verify that eOPN3-mediated presynaptic silencing can be utilized in a behavioral setting, eOPN3 was applied to suppress auditory input to the amygdala during recall and renewal of auditory-cued fear conditioning [FIG. 6A, (Tovote, et al., 2015)]. The amygdala plays a central role in the acquisition as well as the recall of the conditioned freezing response (LeDoux, 2000). Thus, inhibition of terminals from the medial geniculate nucleus (MGN) and the auditory cortex (ACtx) during the delivery of the conditioned stimulus (CS) should suppress the expression of the cue-triggered freezing response (Nabavi, et al., 2014; Kim & Cho, 2017). To this end, mice were bilaterally injected with AAV encoding eOPN3 or a fluorophore-only control vector into the MGN and the ACtx and 200 μm-diameter optical fibers were implanted above the amygdala (FIG. 6B). Following 8-10 weeks of recovery, mice underwent fear conditioning in context A (FIG. 6C), leading to similar freezing levels to the last tone as measured for control mice (FIG. 6D, “Acquisition”; unpaired two-sample Wilcoxon test, p=0.90). During the extinction session, photoactivation of eOPN3 led to a trend of reduced tone-evoked freezing during the first tone block compared with the control group (FIG. 6D, “Extinction”). In contrast, freezing levels were similar between the two groups during a cued recall experiment when no light was delivered (FIG. 6D, “Recall”). To further test the impact of eOPN3 activation in auditory afferents to the BLA on cue-evoked freezing responses, a fear renewal experiment was performed (FIG. 6D, “Renewal”). In this experimental session, the mice were allowed to habituate for 10 minutes in context A. Following 10 minutes in context A, both eOPN3-expressing as well as control mice showed low freezing rates (22.9% and 23.1% for eOPN3- and ctrl-mice, respectively). All mice were then exposed to the CS for 5 additional minutes. Activation of eOPN3 in auditory afferents to the amygdala was initiated 5 minutes after the beginning of the experiment and persisted throughout the 5 minutes of tone presentation, allowing assessing the effects of this manipulation both on contextual and cued freezing. To activate eOPN3, 1 s light pulses (10 mW) were bilaterally delivered every 10 seconds. Activation of eOPN3 had no effect on context-evoked freezing. However, eOPN3-expressing mice showed reduced CS-evoked freezing during the 5 minutes of CS presentation (FIG. 6D, “Renewal”), indicating that eOPN3 can effectively suppress the activity of MGN and ACtx terminals in the amygdala. Across individual mice, freezing responses were negatively correlated with eOPN3-mScarlet expression levels in the experimental group but showed no correlation with eYFP expression levels in the control group (FIG. 6E).

Taken together, the results demonstrate that eOPN3 can be used for synaptic terminal inhibition in behaving animals, with a high light-sensitivity and a precise timed onset.

### Example 4

### Exogenous Expression of Bistable Type II Opsins and Switchable Control of Presynaptic Transmission

As shown for OPN3, cloning of the opsin-mScarlet in frame with an ER-export signal and a membrane trafficking sequence (yielding eOPN3) increased exogenous expression and membrane targeting in cultured hippocampal neurons. Hence, other bistable type-II opsins were cloned with the same strategy to test for functional activation of the G coupled Gi/o pathway. As with the eOPN3, the ability to evoke GIRK mediated photocurrents, when co-transfected with the G protein-gated potassium channel GIRK2.1, was tested. PdCO2, LcPP, medakaTMT1A, zPP1 and pPP2 where well tolerated and coupling to GIRK was achieved by ultraviolet to blue light (FIG. 7A). Moreover, most bi-stable opsins could be completely deactivated with application of red shifted light for 5-10 seconds, enabling bimodal control over the opsins by light (FIG. 7A, green and yellow bars).

For neuronal cultures expressing PdCO2, blue light activation led to GIRK mediated photocurrents as well (FIG. 7B, upper trace), demonstrating the ability to also activate endogenous GIRK channels by PdCO2 activation. In addition, the normalized mEPSC frequency was reduced to about 50% when PdCO2 was once activated and only returned to the baseline frequency when PdCO2 was inactivated with green light, 100 s post activation (FIG. 7B, lower trace and plots). Notably, mEPSC frequency reduction duration exceeded the occurrence of GIRK mediated photocurrents, demonstrating the ability for long term inhibition of synaptic transmission that is not coupled to GIRK activity.

Due to the bi-stable nature of these opsins, they can be activated, inactivated and reactivated again. As shown before (FIGS. 7A-B), blue and green light were successively applied for activation and deactivation, respectively (FIG. 7C, upper panel). In successive trials, PdCO2 could reversibly and repetitively inhibit synaptic transmission (FIG. 7C).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

(other references are cited throughout the application)
Airan, R. D., Thompson, K. R., Fenno, L. E., Bernstein, H., Deisseroth, K., 2009. Temporally precise in vivo control of intracellular signalling. Nature 458, 1025-1029. doi:10.1038/nature07926
Basu, J., Zaremba, J. D., Cheung, S. K., Hitti, F. L., Zemelman, B. V., Losonczy, A., Siegelbaum, S. A., 2016. Gating of hippocampal activity, plasticity, and memory by entorhinal cortex long-range inhibition. Science (New York, N.Y 351, aaa5694-aaa5694. doi:10.1126/science.aaa5694
Creed, M., Pascoli, V. J., Luscher, C., 2015. Addiction therapy. Refining deep brain stimulation to emulate optogenetic treatment of synaptic pathology. Science (New York, N.Y 347, 659-664. doi:10.1126/science.1260776
Dobrunz, L. E., Huang, E. P., Stevens, C. F., 1997. Very short-term plasticity in hippocampal synapses. Proceedings of the National Academy of Sciences of the United States of America 94, 14843-14847.
Isoldi, M. C., Rollag, M. D., Castrucci, A. M. de L., Provencio, I., 2005. Rhabdomeric phototransduction initiated by the vertebrate photopigment melanopsin. Proceedings of the National Academy of Sciences of the United States of America 102, 1217-1221. doi:10.1073/pnas.0409252102
Klavir, O., Prigge, M., Sarel, A., Paz, R., Yizhar, O., 2017. Manipulating fear associations via optogenetic modulation of amygdala inputs to prefrontal cortex. Nat. Neurosci. 20, 836-844. doi:10.1038/nn.4523
Koyanagi, M., Takada, E., Nagata, T., Tsukamoto, H., Terakita, A., 2013. Homologs of vertebrate Opn3 potentially serve as a light sensor in nonphotoreceptive tissue. Proc Natl Acad Sci USA 110, 4998-5003. doi:10.1073/pnas. 1219416110
Mahn, M., Gibor, L., Patil, P., Cohen-Kashi Malina, K., Oring, S., Printz, Y., Levy, R., Lampl, I., Yizhar, O., 2018. High-efficiency optogenetic silencing with soma-targeted anion-conducting channelrhodopsins. Nat Commun 9, 4125. doi:10.1038/s41467-018-06511-8
Mahn, M., Prigge, M., Ron, S., Levy, R., Yizhar, O., 2016. Biophysical constraints of optogenetic inhibition at presynaptic terminals. Nat. Neurosci. 19, 554-556. doi:10.1038/nn.4266
Nabavi, S., Fox, R., Proulx, C. D., Lin, J. Y., Tsien, R. Y., Malinow, R., 2014. Engineering a memory with LTD and LTP. Nature 511, 348-352.
Segal, M. M., 1991. Epileptiform activity in microcultures containing one excitatory hippocampal neuron. J Neurophysiol 65, 761-770. doi:10.1152/jn.1991.65.4.761
Stachniak, T. J., Ghosh, A., Sternson, S. M., 2014. Chemogenetic synaptic silencing of neural circuits localizes a hypothalamus→midbrain pathway for feeding behavior. Neuron 82, 797-808. doi:10.1016/j.neuron.2014.04.008
Sternson, S. M., Roth, B. L., 2014. Chemogenetic tools to interrogate brain functions. Annu. Rev. Neurosci. 37, 387-407. doi:10.1146/annurev-neuro-071013-014048
Wiegert, J. S., Mahn, M., Prigge, M., Printz, Y., Yizhar, O., 2017. Silencing Neurons: Tools, Applications, and Experimental Constraints. Neuron 95, 504-529. doi:10.1016/j.neuron.2017.06.050
Wu, L. G., Saggau, P., 1994. Adenosine inhibits evoked synaptic transmission primarily by reducing presynaptic calcium influx in area CA1 of hippocampus. Neuron 12, 1139-1148.
Yizhar, O., Fenno, L. E., Davidson, T. J., Mogri, M., Deisseroth, K., 2011. Optogenetics in neural systems. Neuron 71, 9-34. doi:10.1016/j.neuron.2011.06.004
Zhu, H., Roth, B. L., 2014. Silencing synapses with DREADDs. Neuron 82, 723-725. doi:10.1016/j.neuron.2014.05.002
Zurawski, Z., Yim, Y. Y., Alford, S., Hamm, H. E., 2019. The expanding roles and mechanisms of G protein-mediated presynaptic inhibition. J. Biol. Chem. 294, 1661-1670. doi:10.1074/jbc.TM118.004163

What is claimed is:

1. A polynucleotide encoding a polypeptide comprising a bistable type II opsin, an ER export signal and a membrane trafficking signal, said ER export signal and said membrane trafficking signal being heterologous to said bistable type II opsin.

2. The polynucleotide of claim 1, wherein said bistable type II opsin is OPN3.

3. The polynucleotide of claim 1, wherein said OPN3 is mosquito OPN3 (MosOpn3).

4. The polynucleotide of claim 1, wherein said ER export signal and said said membrane trafficking signal are of a protein expressed in neuronal cells.

5. The polynucleotide of claim 1, wherein said ER export signal and said membrane trafficking signal enable trafficking to axonal presynaptic terminals.

6. The polynucleotide of claim 1, wherein said ER export signal and said membrane trafficking signal is of a Kir2.1 polypeptide.

7. The polynucleotide of claim 1, wherein said ER export signal comprises SEQ ID NO: 2 and said membrane trafficking signal comprises SEQ ID NO: 1.

8. A method of generating a G-protein signaling in a cell, the method comprising exposing a cell expressing the polynucleotide of claim 1 to light in a wavelength that activates the polypeptide.

9. The method of claim 8, wherein said cell is a mammalian cell.

10. The method of claim 8, wherein said cell is a human cell.

11. The method of claim 8, wherein said cell is a neuron.

12. The method of claim 8, wherein said G-protein signaling is a Gi/o signaling.

13. The method of claim 8, wherein said method is effected in-vitro or ex-vivo.

14. The method of claim 8, wherein said method is effected in-vivo.

15. The method of claim 8, wherein said wavelength is 350-650 nm.

16. A method of generating a G-protein signaling in a cell in a subject in need thereof, the method comprising:
(a) administering to the subject the polynucleotide of claim 1; and
(b) exposing a tissue region of said subject comprising said polynucleotide to light in a wavelength that activates said polypeptide.

17. A method of treating a disease that can be alleviated by this therapy in a subject in need thereof, the method comprising:
(a) administering to the subject a therapeutically effective amount of the polynucleotide of claim 1; and
(b) exposing a tissue region of said subject comprising said polynucleotide, to light in a wavelength that activates said polypeptide,
thereby treating the disease in the subject.

18. The method of claim 17, wherein said disease is associated with excitability of excitable tissues.

19. The method of claim 18, wherein said disease is a neurological disease.

20. The method of claim 19, wherein said neurological disease is selected from the group consisting of Parkinson, pain, epilepsy, depression, essential tremor, motor neuron disease, dystonia, obsessive compulsive disorder, addiction, schizophrenia, post-traumatic stress disorder, panic disorder and anxiety.
